SEQUENCE LISTING (38 sequences)

SEQ ID NO: 1 - protein, 147 aa, synthetic construct
MAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQKGGGGSGGGGSGAHWQFNALTVR

SEQ ID NO: 2 - protein, 163 aa, synthetic construct
MAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQKGGGGSGGGGSGLRSKSKKFRRPDIQYPDATDEDITSHM

SEQ ID NO: 3 - protein, 150 aa, synthetic construct
MAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQKGGGGSGGGGSTGLPVGVGYVVTVLT

SEQ ID NO: 4 - protein, 140 aa, synthetic construct
MAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQKGGGGSGGGGSKKKKK

SEQ ID NO: 5 - protein, 12 aa, synthetic construct
GAHWQFNALTVR

SEQ ID NO: 6 - protein, 15 aa, synthetic construct
TGLPVGVGYVVTVLT

SEQ ID NO: 7 - protein, 28 aa, Homo sapiens
GLRSKSKKFRRPDIQYPDATDEDITSHM

SEQ ID NO: 8 - protein, 5 aa, synthetic construct
KKKKK

SEQ ID NO: 9 - protein, 125 aa, Mus sp.
MAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 10 - protein, 8 aa, synthetic construct
GGGSGGGS

SEQ ID NO: 11 - protein, 6 aa, synthetic construct
HHHHHH

SEQ ID NO: 12 - protein, 6 aa, synthetic construct
ENLYFQ

SEQ ID NO: 13 - protein, 124 aa, Mus sp.
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 14 - protein, 124 aa, Mus sp.
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 15 - protein, 127 aa, Homo sapiens [sequence omitted]

SEQ ID NO: 16 - protein, 127 aa, Homo sapiens
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQTITFESFKENLKDFLLVIPFDCWEPVQE

SEQ ID NO: 17 - protein, 338 aa, Homo sapiens
MSLSAFTLFLALIGGTSGQYYDYDFPLSIYGQSSPNCAPECNCPESYPSAMYCDELKLKSVPMVPPGIKYLYLRNNQIDHIDEKAFENVTDLQWLILDHNLLENSKIKGRVFSKLKQLKKLHINHNNLTESVGPLPKSLEDLQLTHNKITKLGSFEGLVNLTFIHLQHNRLKEDAVSAAFKGLKSLEYLDLSFNQIARLPSGLPVSLLTLYLDNNKISNIPDEYFKRFNALQYLRLSHNELADSGIPGNSFNVSSLVELDLSYNKLKNIPTVNENLENYYLEVNQLEKFDIKSFCKILGPLSYSKIKHLRLDGNRISETSLPPDMYECLRVANEVTLN

SEQ ID NO: 18 - protein, 263 aa, Mus sp.
MSLHPVILLVLVLCLGWKINTQEGSLPDITIFPNSSLMISQGTFVTVVCSYSDKHDLYNMVRLEKDGSTFMEKSTEPYKTEDEFEIGPVNETITGHYSCIYSKGITWSERSKTLELKVIKENVIQTPAPGPTSDTSWLKTYSIYIFTVVSVIFLLCLSALLFCFLRHRQKKQGLPNNKRQQQRPEERLNLATNGLEMTPDIVADDRLPEDRWTETWTPVAGDLQEVTYIQLDHHSLTQRAVGAVTSQSTDMAESSTYAAIIRH

SEQ ID NO: 19 - protein, 287 aa, Homo sapiens
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 20 - protein, 144 aa, Homo sapiens
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLY

SEQ ID NO: 21 - protein, 152 aa, Homo sapiens
MSPHLTALLGLVLCLAQTIHTQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAP

SEQ ID NO: 22 - protein, 33 aa, synthetic construct
GCPRPRGDNPPLTCSQDSDCLAGCVCGPNGFCG

SEQ ID NO: 23 - protein, 33 aa, synthetic construct
GCPRPRGDNPPLTCKQDSDCLAGCVCGPNGFCG

SEQ ID NO: 24 - protein, 127 aa, Homo sapiens
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 25 - protein, 76 aa, synthetic construct
  VARIANT 2..76: note = This region may encompass 1-15 GGGGS repeating units
SGGGGSGGGGSGGGGSGGGGSGGGGSGGGGSGGGGSGGGGSGGGGSGGGGSGGGGSGGGGSGGGGSGGGGSGGGGS

SEQ ID NO: 26 - protein, 75 aa, synthetic construct
  VARIANT 1..75: note = This sequence may encompass 1-15 GGGGS repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 27 - protein, 60 aa, synthetic construct
  VARIANT 1..60: note = This sequence may encompass 1-15 GGGS repeating units
GGGSGGGSGGGSGGGSGGGSGGGSGGGSGGGSGGGSGGGSGGGSGGGSGGGSGGGSGGGS

SEQ ID NO: 28 - protein, 105 aa, synthetic construct
  VARIANT 1..105: note = This sequence may encompass 1-15 GGGGSSS repeating units
GGGGSSSGGGGSSSGGGGSSSGGGGSSSGGGGSSSGGGGSSSGGGGSSSGGGGSSSGGGGSSSGGGGSSSGGGGSSSGGGGSSSGGGGSSSGGGGSSSGGGGSSS

SEQ ID NO: 29 - protein, 75 aa, synthetic construct
  VARIANT 1..75: note = This sequence may encompass 1-15 SGGGG repeating units
SGGGGSGGGGSGGGGSGGGGSGGGGSGGGGSGGGGSGGGGSGGGGSGGGGSGGGGSGGGGSGGGGSGGGGSGGGG

SEQ ID NO: 30 - protein, 79 aa, synthetic construct
  VARIANT 5..79: note = This region may encompass 1-15 SGGGG repeating units
GGGGSGGGGSGGGGSGGGGSGGGGSGGGGSGGGGSGGGGSGGGGSGGGGSGGGGSGGGGSGGGGSGGGGSGGGGSGGGG

SEQ ID NO: 31 - protein, 75 aa, synthetic construct; identical to SEQ ID NO: 26
  VARIANT 1..75: note = This sequence may encompass 1-15 GGGGS repeating units

SEQ ID NO: 32 - protein, 22 aa, synthetic construct
GGGGSGGGGSGAHWQFNALTVR

SEQ ID NO: 33 - protein, 38 aa, synthetic construct
GGGGSGGGGSGLRSKSKKFRRPDIQYPDATDEDITSHM

SEQ ID NO: 34 - protein, 25 aa, synthetic construct
GGGGSGGGGSTGLPVGVGYVVTVLT

SEQ ID NO: 35 - protein, 15 aa, synthetic construct
GGGGSGGGGSKKKKK

SEQ ID NO: 36 - protein, 127 aa, synthetic construct [sequence omitted]

SEQ ID NO: 37 - protein, 127 aa, synthetic construct; identical to SEQ ID NO: 16

SEQ ID NO: 38 - protein, 127 aa, synthetic construct; identical to SEQ ID NO: 24

(19) United States
(12) Patent Application Publication
(10) Pub. No.: US 2026/0139022 A1
(43) Pub. Date: May 21, 2026

(54) COMPOSITIONS INCLUDING TUMOR-BINDING GMCSF FUSION PROTEINS AND METHODS OF USING THE SAME TO TREAT SOLID TUMORS

(71) Applicant: University of Kansas, Lawrence, KS (US)

(72) Inventors: Cory BERKLAND, Lawrence, KS (US); Brandon DEKOSKY, Cambridge, MA (US); Amy LAFLIN, Andover, KS (US); Marcus Laird FORREST, Lawrence, KS (US); Aparna Raghavachar CHAKRAVARTI, Lawrence, KS (US)

(21) Appl. No.: 18/705,864

(22) PCT Filed: Nov. 2, 2022
(86) PCT No.: PCT/US2022/048755

§ 371 Date: Apr. 29, 2024

Related U.S. Application Data
(60) Provisional application No. 63/275,224, filed on Nov. 3, 2021.

(51) Int. Cl. C07K 14/535; A61K 38/00; A61K 45/06; A61P 35/00; C07K 7/08

(52) U.S. Cl. CPC C07K 14/535; A61K 45/06; A61P 35/00; C07K 7/08; A61K 38/00; C07K 2319/02; C07K 2319/20; C07K 2319/70

(57) ABSTRACT

The present disclosure relates generally to compositions comprising variant granulocyte macrophage-colony-stimulating factor (GMCSF) fusion polypeptides that are configured to bind to a tumor extracellular matrix (ECM) component, and methods for using the same to treat solid tumors.

### CROSS-REFERENCE TO RELATED APPLICATIONS

This application claims the benefit of and priority to U.S. Provisional Appl. No. 63/275,224, filed Nov. 3, 2021, the disclosure of which is incorporated by reference herein in its entirety.

### TECHNICAL FIELD

The present technology relates generally to compositions comprising variant granulocyte macrophage-colony-stimulating factor (GMCSF) fusion polypeptides that are configured to bind to a tumor extracellular matrix (ECM) component, and methods for using the same to treat solid tumors.

### BACKGROUND

The following description of the background of the present technology is provided simply as an aid in understanding the present technology and is not admitted to describe or constitute prior art to the present technology.

The tumor microenvironment produces signals and creates cell phenotypes that allow evasion of the host immune response leading to adaptive immune resistance (‘Cold’ tumor). Identification of IT immunostimulants as an alternative therapy in the treatment of solid tumors is a rapidly rising field of interest. Since the immune cell makeup of a tumor ultimately determines the clinical outcome, striking the right balance between anti-tumor and pro-tumor responses can be a major contributing factor of therapeutic efficacy.

Systemic administration of immunostimulants has several drawbacks such as poor drug trafficking, systemic toxicity, off-target effects, poor retention at tumor sites, and rapid renal/hepatic clearance due to small molecular size.

### SUMMARY OF THE PRESENT TECHNOLOGY

In one aspect, the present disclosure provides a fusion polypeptide comprising a mammalian granulocyte macrophage-colony-stimulating factor (GMCSF) polypeptide operably linked to a tumor binding peptide, wherein the tumor binding peptide is configured to bind to a tumor extracellular matrix (ECM) component. In certain embodiments, the tumor ECM component is hyaluronic acid, fibronectin, or collagen. Additionally or alternatively, in some embodiments, the tumor binding peptide is linked to the N-terminus or C-terminus of the mammalian GMCSF polypeptide.

In some embodiments, the mammalian GMCSF polypeptide is murine GMCSF or human GMCSF. In certain embodiments, the mammalian GMCSF polypeptide comprises an amino acid sequence selected from the group consisting of sargramostim, molgramostim, and regramostim. Additionally or alternatively, in some embodiments, the mammalian GMCSF polypeptide comprises the amino acid sequence of any one of SEQ ID NOs: 9, 13-16 or 24. The mammalian GMCSF polypeptide may or may not comprise an endogenous or heterologous signal peptide sequence.

Additionally or alternatively, in some embodiments, the mammalian GMCSF polypeptide is fused to the tumor binding peptide directly or via a peptide linker. Examples of peptide linkers include a gly-ser polypeptide linker, a glycine-praline polypeptide linker, or a proline-alanine polypeptide linker. In certain embodiments, the peptide linker is selected from the group consisting of S(G₄S)ₙ, (G₄S)ₙ, (G₃S)ₙ, (G₄S₃)ₙ, (SG₄)ₙ or G+ (SG+)ₙ, wherein n is 1, 2, 3, 4, 5, 6, 7, 8, 9, 10, 11, 12, 13, 14, or 15.

In any of the preceding embodiments, the tumor binding peptide comprises a collagen-binding domain, a hyaluronic acid binding peptide (HABP), integrin-binding polypeptide, or a fibronectin binding peptide (FnBP). In some embodiments, the collagen-binding domain comprises a proteoglycan. Examples of such proteoglycans include, but are not limited to, decorin, biglycan, testican, bikunin, fibromodulin, lumican, chondroadherin, keratin, ECM2, epiphycan, asporin, PRELP, keratocan, osteoadherin, opticin, osteoglycan, nyctalopin, Tsukushi, podocan, podocan-like protein 1 versican, perlecan, nidogen, neurocan, aggrecan, osteopontin, and brevican. Additionally or alternatively, in certain embodiments, the collagen-binding domain comprises a class I small leucine-rich proteoglycan (SLRP), a class II SLRP, a class III SLRP, a class IV SLRP, or a class V SLRP. In any of the above embodiments, the tumor binding peptide comprises the amino acid sequence selected from the group consisting of SEQ ID NOs: 5-8 and 17-23.

Additionally or alternatively, in some embodiments, the fusion polypeptide comprises an amino acid sequence selected from the group consisting of SEQ ID NOs: 1-4.

In one aspect, the present disclosure provides a pharmaceutical composition comprising any and all embodiments of the fusion polypeptide described herein, and a pharmaceutically acceptable carrier.

In another aspect, the present disclosure provides a method for treating cancer or inhibiting tumor growth in a patient in need thereof, comprising administering to the patient a therapeutically effective amount of any and all embodiments of the fusion polypeptide described herein or any and all embodiments of the pharmaceutical compositions described herein.

In yet another aspect, the present disclosure provides a method for enhancing responsiveness of a cancer patient to immune checkpoint inhibitor therapy comprising administering to the patient a therapeutically effective amount of any and all embodiments of the fusion polypeptide described herein or any and all embodiments of the pharmaceutical compositions described herein; and administering to the patient a therapeutically effective amount of an immune checkpoint inhibitor.

In some embodiments, the cancer is a solid tumor. Examples of cancers include, but are not limited to, melanoma, mesothelioma, pancreatic cancer, glioblastoma, breast cancer, ovarian cancer, lung cancer, colorectal cancer, or prostate cancer.

Additionally or alternatively, in some embodiments, the fusion polypeptide or the pharmaceutical composition is administered intratumorally, orally, topically, intranasally, systemically, intravenously, subcutaneously, intraperitoneally, intradermally, intraocularly, iontophoretically, transmucosally, or intramuscularly.

Additionally or alternatively, in certain embodiments, the method further comprises separately, sequentially or simultaneously administering one or more additional therapeutic agents to the subject. The one or more additional therapeutic agents may comprise an immune checkpoint inhibitor, a chemotherapeutic agent and/or a radiotherapeutic agent. In certain embodiments, the immune checkpoint inhibitor is an anti-PD1 antibody or an anti-PD-L1 antibody. Examples of immune checkpoint inhibitor include, but are not limited to, ipilimumab, pembrolizumab, nivolumab, atezolizumab, avelumab, durvalumab, pidilizumab, AMP-224, MPDL3280A, MDX-1105, MEDI-4736, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, or any combination thereof.

In any and all embodiments of the methods disclosed herein, administration of the fusion polypeptide or the pharmaceutical composition reduces the incidence of and/or mitigates systemic immune-related adverse events (IRAEs) in the patient.

Also disclosed herein are kits comprising any and all embodiments of the fusion polypeptide described herein or any and all embodiments of the pharmaceutical compositions described herein, and instructions for using the same to treat or prevent cancer.

### BRIEF DESCRIPTION OF THE DRAWINGS

FIGS. 1A-1D. Soluble expression of eGMCSF variants in E. coli. FIG. 1A: Structure of PeT9a-eGMCSF expression plasmid. FIG. 1B: Representative structure of engineered variant GMCSF fusion polypeptides of the present technology. FIG. 1C: Representative RP-HPLC chromatograph for the purified eGMCSF variants (C4 Column, Method: 20-45% B, 20 minutes; Solvents: A-H20 (+0.05% TFA), B-ACN (+0.05% TFA)). FIG. 1D: SDS-PAGE analysis and protein yields of purified eGMCSF variants per 4 L of E. coli culture. FIG. 1E: Schematic representing the synthesis of tumor-retentive eGMCSF via recombinant protein engineering. FIG. 1F: Endotoxin levels (EU/mL) in the different protein formulations. All data are reported as mean±standard deviation (n=3). *=p<0.05, **=p<0.01, ***=p<0.001, ****=p<0.0001.

FIGS. 2A-2B. Biophysical characterization of eGMCSF. FIG. 2A. Far-UV CD spectra of expressed eGMCSFs compared with standard unmodified mGMCSF. Positive peak at 193 nm and negative peaks at 208 nm and 222 nm in the CD spectra depicts α-helical structure in the eGMCSF variants. Percentage of α-helical character calculated using Bestsel software. FIG. 2B. Dynamic light scattering (DLS) of eGMCSF variants of the present technology.

FIG. 3: Immunomodulatory effects eGMCSF. Dose-dependent cell proliferative activity of eGMCSF variants performed on JAWS II DC cell line. eGMCSF-HAₚ. eGMCSF-CBMₚ, eGMCSF-Fnₚ and eGMCSF-Lys₅ exhibit a proliferative activity on DCs in a manner similar to unmodified mGMCSF. Statistical analysis performed against the control group (n=4). Data are reported as mean±standard deviation *=p<0.05, **=p<0.01, ***=p<0.001, ****=p<0.0001

FIGS. 4A-4C: Intratumoral (IT) retention of eGMCSF. FIG. 4A. Schematic depicting the synthesis of the dispersed gel model to simulate IT retention of the corresponding eGMCSF protein. FIG. 4B. eGMCSF-HAₚ and eGMCSF-Lys₅ displayed a greater binding to HA in comparison to unmodified mGMCSF. eGMCSF-CBMₚ, eGMCSF-Fnₚ variants displayed a greater binding to Collagen I & Fibronectin, respectively compared to unmodified mGMCSF. FIG. 4C. The tumor binding peptides (TBPs) competitively inhibited the binding of eGMCSF to its respective tumor ECM component (Competitive Inhibition). The driving force behind the tumor binding properties of the eGMCSF variants is the presence of TBP in its structure. Data is reported as mean±SD. (n=3)

FIGS. 5A-5D. Ex vivo intratumoral (IT) retention of eGMCSF. FIG. 5A. Study design for generation of ex vivo HNSCC tumors from murine models. FIG. 5B. Schematic depicting the IT injection of FITC-labeled eGMCSF in the ex vivo tumors. FIG. 5C. Average tumor volume between the different test groups. The distribution of resected tumors between the groups was done to ensure a negligible difference in average tumor volume. FIG. 5D. eGMCSF-HAₚ, eGMCSF-Lys₅, eGMCSF-CBMₚ, and eGMCSF-Fnₚ displayed a greater binding to tumor tissue in comparison to unmodified mGMCSF over the course of 21 days. Data is reported as mean±SD. (n=3)

FIGS. 6A-6D. In vivo therapeutic efficacy of eGMCSF in colon cancer model. FIG. 6A. Schematic of IT treatment. Drugs were administered when tumors reached ˜100 mm³, generally 10-12 days after tumor inoculation. FIG. 6B. Tumor growth curves for the dose escalation study treatment groups. Each data point is the mouse's tumor volume as a fold increase from the first day of tumor inoculation. (n=3). FIG. 6C. Tumor growth curves for the monotherapy treatment groups. FIG. 6D. Tumor growth curves for the combination therapy with a CPI-anti-PD1. 2-way ANOVA with Sidak's multiple comparisons test (n=5) * vs respective Vehicle group, † vs respective mGMCSF groups.

FIGS. 7A-7E. In vivo therapeutic efficacy of eGMCSF in colon cancer model. Comparison between monotherapy and combination therapy for each eGMCSF variant. Mice were treated with 4% mannitol served as vehicle.

FIG. 8. In vivo immune cell infiltration in eGMCSF treated colon cancer tumors. Comparison between monotherapy and combination therapy for each eGMCSF variant. Mice were treated with 4% mannitol served as vehicle. Tumors sections are stained to visualize cell nuclei (blue), CD11b (dendritic cells, monocytes, granulocytes, macrophages, NK cells, T cells, B cells; red), CD11c (dendritic cells; pink). (Scale bar: 2 mm, 10× magnification)

FIGS. 9A-9F. In vivo systemic toxicity after first drug injection. Mouse serum cytokines 2 hours after first drug injection. Mice treated with unmodified mGMCSF, and 4% mannitol served as controls. All drugs were tested as a monotherapy (black) and as a combination therapy with CPI-anti PD-1 (grey). 2-way ANOVA with Sidak's multiple comparisons test (n=5) * vs respective Vehicle group, † vs respective mGMCSF groups.

FIGS. 10A-10F. In vivo systemic toxicity after last drug injection. Mouse serum cytokines 2 hours after last drug injection. Mice treated with unmodified mGMCSF, and 4% mannitol served as controls. All drugs were tested as a monotherapy (black) and as a combination therapy with CPI-anti PD-1 (grey). 2-way ANOVA with Sidak's multiple comparisons test (n=5) * vs respective Vehicle group, † vs respective mGMCSF groups.

FIG. 11. CD analysis of free tumor binding peptides (TBPs). All three TBPs possess a random coil structure.

FIG. 12. Fluorescent image of ex vivo tumors 23 days post IT retention study. To negate tissue autofluorescence each tumor's fluorescent intensity is normalized to that of the vehicle control using Image J. Superior retention of FITC-labeled eGMCSF was observed in HNSCC tumors in comparison to unmodified mGMCSF and vehicle controls

FIG. 13. Individual tumor growth curve for the in vivo therapeutic efficacy study: monotherapy

FIG. 14. Individual tumor growth curve for the in vivo therapeutic efficacy study: combination therapy.

FIG. 15. Proposed mechanism of action of variant granulocyte macrophage-colony-stimulating factor (GMCSF) fusion polypeptides of the present technology.

FIG. 16. Amino acid sequences of molgramostim, regramostim and sargramostim.

FIG. 17. Comparison of the diffusion (release) of eGM-CSFs of the present technology with or without the His tag.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure provides intratumoral (IT) cytokine immunotherapy methods that exhibit superior retention and immune stimulation at the injection site, thereby providing a safer and more efficacious treatment strategy for solid tumors in comparison to current treatment options. Presently available cancer therapies rely on the use of immunostimulants to selectively activate the host's immune system against the tumor cells. So far, a variety of therapeutic agents, including cytokines, checkpoint inhibitors (CPIs), oncolytic viruses, and monoclonal antibodies (mAbs), have transformed the landscape of cancer immunotherapy by targeting local and metastatic tumors. However, the success rate in patients remains fairly low, primarily due to the traditional intravenous (IV) delivery of these immunostimulants. IV administration causes the therapeutic to enter systemic circulation, with the drug needing to traverse various biological tissue barriers before reaching the target tumor tissue. This limits the drugs' therapeutic potency since only a minimal therapeutic dose reaches the cancerous site, while the majority persists in the systemic circulation, causing toxicity. An additional contributing factor that renders these treatments inefficient is the resistance of nonimmunogenic ‘cold’ tumors to immunotherapy.

Recent publications have explored the IT delivery of a cytokine-granulocyte macrophage-colony-stimulating factor (GMCSF) in various clinical trials for the treatment of solid tumors like melanoma and mesothelioma. GMCSF functions by inducing the development and maturation of antigen-presenting cells (APCs) to tip the immune system towards anti-tumor T cell responses by promoting lymphocyte infiltration into tumor tissue. While these IT cytokine therapies yielded good tumor clearance, several patients displayed an increased risk of adverse events and side effects due to systemic cytokine exposure possibly caused by the poor retention of GMCSF within the tumor tissue.

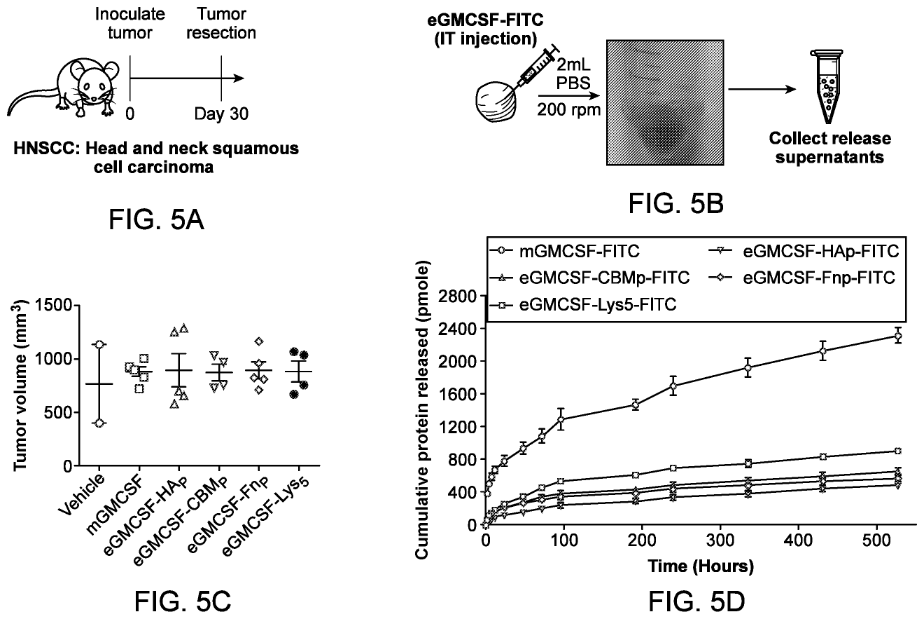

The inherent ECM heterogeneity amongst patient tumors can impede the clinical efficacy of these tumor-retentive immunostimulants. To overcome the downfalls of targeting a single tumor target, disclosed herein are multiple eGMCSFs that bind to the most prevalent ECM tumor components. The eGMCSF drug can be distinctively utilized to treat the tumors spatiotemporally and maximize the therapeutic benefits of the engineered cytokine. The variant GMCSF fusion polypeptides of the present technology are retained in the solid tumor following IT administration, with limited systemic exposure and elicits local inflammation and immune infiltration due to the establishment of a local chemokine gradient (FIG. 5).

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form and a second plurality are in the L form. Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

As used herein, the terms “cancer” (or “cancerous”), “hyperproliferative,” and “neoplastic” cells refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g, by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

The term “fusion polypeptide” as used herein, refers to a protein that is created by joining two or more elements, components, or domains and/or polypeptides to create a larger polypeptide. As used herein, the terms “linked,” “operably linked,” “fused” or “fusion”, are used interchangeably, and refers to the joining together of two or more elements, components, domains and/or polypeptides within a fusion polypeptide that allow for at least one element, component, domain and/or polypeptide to have at least a portion of the biological function or cellular activity when expressed in the fusion polypeptide as when expressed in its natural state and/or without the linkage. The joining together of the two more elements or components or domains can be performed by whatever means known in the art including chemical conjugation, noncovalent complex formation or recombinant means. Methods of chemical conjugation (e.g., using heterobifunctional crosslinking agents) are known in the art. Thus, the elements, components, domains and/or polypeptides can be joined by covalent bonds (e.g., peptide bonds) or non-covalent bonds. The elements, components, domains and/or polypeptides can be joined by peptide bond formation in the ribosome during translation or post-translationally.

As used herein, the term “gene” means a segment of DNA that contains all the information for the regulated biosynthesis of an RNA product, including promoters, exons, introns, and other untranslated regions that control expression.

As used herein, “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter-none; strand-both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the terms “individual”, “patient”, or “subject” can be an individual organism, a vertebrate, a mammal, or a human. In some embodiments, the individual, patient or subject is a human.

The term “integrin-binding polypeptide” refers to a polypeptide which includes an integrin-binding domain or loop within a knottin polypeptide scaffold. The integrin binding domain or loop includes at least one RGD peptide. In certain embodiments, the RGD peptide is recognized by αᵥβ₁, αᵥβ3, αᵥB₅, αᵥβ6, and α₅β₁ integrins. In certain embodiments the RGD peptide binds to a combination of αᵥβ₁, αᵥβ₃, αᵥβ₅, αᵥβ₆, and α₅β₁ integrins. These specific integrins are found on tumor cells and their vasculature and are therefore the targets of interest.

The term “kd”, as used herein, refers to the dissociation rate constant of a particular protein-protein interaction. This value is also referred to as the k_{off} value.

The term “kₐ”, as used herein, refers to the association rate constant of a particular protein-protein interaction. This value is also referred to as the kₒₙ value.

The term “KD” (M), as used herein, refers to the dissociation equilibrium constant of a particular protein-protein interaction. KD=kd/kₐ. In some embodiments, the affinity of a protein (e.g., binding domain) is described in terms of the KD for an interaction between two proteins. For clarity, as known in the art, a smaller KD value indicates a higher affinity interaction, while a larger KD value indicates a lower affinity interaction.

As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together. In certain embodiments, the linker comprises amino acids having the sequence (GGGGS)ₙ, wherein n is at least 1, 2, 3, 4, 5, 6, 7, 8, 9, 10, 11, 12, 13, 14, or 15.

As used herein, the term “nucleic acid” or “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, Pa.).

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, “prevention”, “prevent”, or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample. As used herein, preventing cancer, includes preventing or delaying the initiation of symptoms of cancer. As used herein, prevention of cancer also includes preventing a recurrence of one or more signs or symptoms of cancer.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition. In some embodiments, “inhibiting,” means reducing or slowing the growth of a tumor. In some embodiments, the inhibition of tumor growth may be, for example, by 5% or more, 10% or more, 20% or more, 30% or more, 40% or more, 50% or more, 60% or more, 70% or more, 80% or more, or 90% or more. In some embodiments, the inhibition may be complete.

eGMCSF Fusion Polypeptides of the Present Technology

The engineered GMCSF (eGMCSF) fusion polypeptides of the present technology comprise a mammalian granulocyte macrophage-colony-stimulating factor (GMCSF) polypeptide operably linked to a tumor binding peptide, wherein the tumor binding peptide is configured to bind to a tumor extracellular matrix (ECM) component. The mammalian GMCSF may be linked to the tumor binding peptide directly or via a linker. In some embodiments, the linker is a peptide linker comprising one or more amino acids, typically about 2-20 amino acids, that are described herein or are known in the art. Examples of peptide linker sequences include, but are not limited to, gly-ser polypeptide linkers, glycine-praline polypeptide linkers, and proline-alanine polypeptide linkers. Suitable, non-immunogenic linker peptides include, for example, S(G₄S)ₙ, (G₄S)ₙ, (G₃S)ₙ, (G₄S₃)ₙ, (SG₄) n or G₄ (SG₄)ₙ linker peptides, wherein n is about 1, 2, 3, 4, 5, 6, 7, 8, 9, 10, 11, 12, 13, 14, or 15.

### GMCSF

As used herein, “Granulocyte-macrophage colony-stimulating factor,” (GMCSF) refers to a cytokine that functions by inducing the recruitment and maturation of antigen-presenting cells (APCs) and tipping the immune system towards anti-tumor CD8⁺ T cell responses by promoting lymphocyte infiltration into tumor tissue. In some embodiments, the eGMCSF fusion polypeptide comprises a mammalian GMCSF. In certain embodiments, the mammalian GMCSF is operably linked to a tumor binding peptide that is configured to bind to a tumor extracellular matrix (ECM) component.

In some embodiments, the mammalian GMCSF is a wild-type mammalian GMCSF (e.g., human GMCSF in its precursor form or mature human GMCSF). In some embodiments, the mammalian GMCSF is human or murine GMCSF. Exemplary mammalian GMCSF amino acid sequences are provided below:

Murine GMCSF
(SEQ ID NO: 13)
APTRSPITVTRPWKHVEAIKEALNLLDDMPVTLNEEVEVVSNEFS
FKKLTCVQTRLKIFEQGLRGNFTKLKGALNMTASYYQTYCPPTPE
TDCETQVTTYADFIDSLKTFLTDIPFECKKPVQK
(SEQ ID NO: 14)
APTRSPITVTRPWKHVEAIKEALNLLDDMPVTLNEEVEVVSNEFS
FKKLTCVQTRLKIFEQGLRGNFTKLKGALNMTASYYQTYCPPTPE
TDCETQVTTYADFIDSLKTFLTDIPFECKKPGQK
Human GMCSF
(SEQ ID NO: 15)
APARSPSPSTQPWEHVNAIQEARRLLNLSRDTAAEMNETVEVISE
MFDLQEPTCLQTRLELYKQGLRGSLTKLKGPLTMMASHYKQHCPP
TPETSCATQIITFESFKENLKDFLLVIPFDCWEPVQE
(SEQ ID NO: 16)
APARSPSPSTQPWEHVNAIQEARRLLNLSRDTAAEMNETVEVISE
MFDLQEPTCLQTRLELYKQGLRGSLTKLKGPLTMMASHYKQHCPP
TPETSCATQTITFESFKENLKDFLLVIPFDCWEPVQE
(SEQ ID NO: 24)
APARSPSPSTQPWEHVNAIQEALRLLNLSRDTAAEMNETVEVISE
MFDLQEPTCLQTRLELYKQGLRGSLTKLKGPLTMMASHYKQHCPP
TPETSCATQIITFESFKENLKDFLLVIPFDCWEPVQE

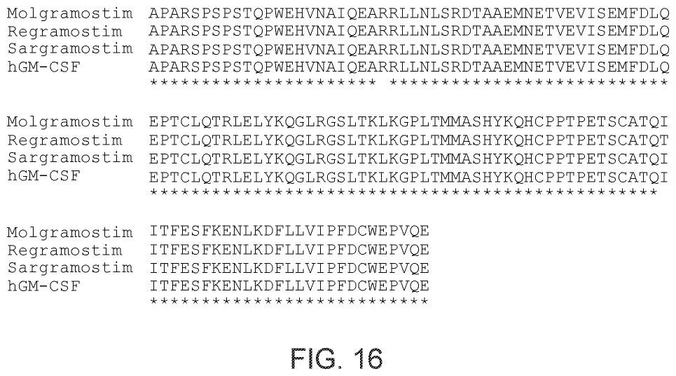

Additionally or alternatively, in certain embodiments, the mammalian GMCSF comprises an amino acid sequence selected from the group consisting of molgramostim, regramostim and sargramostim. See FIG. 16.

In some embodiments, the mammalian GMCSF comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of any one of SEQ ID NOs: 9, 13-16 or 24, or a portion thereof. In some embodiments, the mammalian GMCSF is a variant comprising one or more amino acid substitutions, additions or deletions, optionally two, three, four, five, six, seven, eight, nine, ten or more amino acid substitutions, additions or deletions relative to the amino acid sequence of any one of SEQ ID NOs: 9, 13-16 or 24. In certain embodiments, the mammalian GMCSF is mutated such that it has an altered affinity (e.g., a lower or higher affinity) for the mammalian GMCSF receptor compared with unmodified mammalian GMCSF.

Additionally or alternatively, in some embodiments of the eGMCSF fusion polypeptides of the present technology, the mammalian GMCSF comprises an endogenous or heterologous signal peptide sequence. In other embodiments, the mammalian GMCSF does not comprise a signal peptide sequence.

In some embodiments, the mammalian GMCSF is at the N-terminus of the eGMCSF fusion polypeptides of the present technology. In other embodiments, the mammalian GMCSF is at the C-terminus of the eGMCSF fusion polypeptides of the present technology.

### Tumor Binding Peptide

The tumor binding peptides disclosed herein are configured to bind to a tumor extracellular matrix (ECM) component. The tumor ECM component may be hyaluronic acid, fibronectin, or collagen. In some embodiments, the tumor binding peptide of the eGMCSF fusion polypeptides of the present technology comprise a collagen-binding domain, a HA binding peptide (HABP), integrin-binding polypeptide, or a fibronectin binding peptide (FnBP).

In some embodiments, the tumor binding peptide of the eGMCSF fusion polypeptides of the present technology comprise a collagen-binding domain. In some embodiments, the collagen-binding domain has a molecular weight of about 5-1,000 kDa, about 5-100 kDa, about 10-80 kDa, about 20-60 kDa, about 30-50 kDa, or about 10 kDa, about 20 kDa, about 30 kDa, about 40 kDa, about 50 kDa, about 60 kDa, about 70 kDa, about 80 kDa, about 90 kDa or about 100 kDa. In some embodiments, the collagen-binding domain is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 150 kDa, about 200 kDa, about 300 kDA, about 400 kDa, about 500 kDa, about 600 kDa, about 700 kDa, about 800 kDa, about 900 kDa or about 1,000 kDa.

In some embodiments, the collagen-binding domain is about 10-350, about 10-300, about 10-250, about 10-200, about 10-150, about 10-100, about 10-50, or about 10-20 amino acids in length. In some embodiments, the collagen-binding domain is about 10, about 15, about 20, about 30, about 40, about 50, about 60, about 70, about 80 about 90, about 100, about 120, about 150, about 200, about 250, about 300 or about 350 amino acids in length.

In some embodiments, the collagen-binding domain comprises one or more (e.g, two, three, four, five, six, seven, eight, nine, ten or more) leucine-rich repeats which bind collagen. In some embodiments, the collagen-binding domain comprises a proteoglycan. In some embodiments, the collagen-binding domain comprises a proteoglycan, wherein the proteoglycan is selected from the group consisting of: decorin, biglycan, testican, bikunin, fibromodulin, lumican, chondroadherin, keratin, ECM2, epiphycan, asporin, PRELP, keratocan, osteoadherin, opticin, osteoglycan, nyctalopin, Tsukushi, podocan, podocan-like protein 1 versican, perlecan, nidogen, neurocan, aggrecan, osteopontin, and brevican.

In some embodiments, the collagen-binding domain comprises a class I small leucine-rich proteoglycan (SLRP). In some embodiments, the collagen-binding domain comprises a class II SLRP. In some embodiments, the collagen-binding domain comprises a class III SLRP. In some embodiments, the collagen-binding domain comprises a class IV SLRP. In some embodiments, the collagen-binding domain comprises a class V SLRP. Further description of SLRP classes is provided in Schaefer & Iozzo (2008) J Biol Chem 283 (31): 21305-21309, which is incorporated herein by reference it its entirety. In some embodiments, the collagen-binding domain comprises one or more leucine-rich repeats from a human proteoglycan Class II member of the small leucinerich proteoglycan (SLRP) family. In some embodiments, the SLRP is selected from lumican, decorin, biglycan, fibromodulin, keratin, epiphycan, aspirin, osteopontin, and osteoglycin.

In some embodiments, the collagen-binding domain binds collagen (e.g., collagen type 1 or type 3) with a binding affinity KD value of 0.1-1,000 nM as measured by a suitable method known in the art for determining protein binding affinity, e.g., by ELISA, surface plasmon resonance (BIAcore), FACS analysis, etc. In some embodiments, the collagen-binding domain binds collagen with a binding affinity KD value of 0.1-1.0 nM, 1.0-10 nM. 10-20 nM, 20-30 nM, 30-40 nM, 40-50 nM, 50-60 nM, 70-80 nM, 90-100 nM, 10-50 nM, 50-100 nM, 100-1,000 nM, or 1,000-10,000 nM as determined by a suitable method known in the art. In some embodiments, the eGMCSF fusion polypeptide binds collagen with a binding affinity KD value of 0.1-1.0 nM, 1.0-10 nM, 10-20 nM, 20-30 nM, 30-40 nM, 40-50 nM, 50-60 nM, 70-80 nM, 90-100 nM, 10-50 nM, 50-100 nM, 100-1,000, or 1,000-10,000 nM as determined by a suitable method known in the art. In some embodiments, the collagen-binding domain binds trimeric peptides containing repeated GPO triplets. In some embodiments, the collagen-binding domain binds common collagen motifs in a hydroxyproline-dependent manner.

Exemplary collagen-binding domain sequences are provided below:

| SEQ ID NO: | Description | Sequence |
|---|---|---|
| SEQ ID NO: 7 | osteopontin | GLRSKSKKFRRPDIQYPDATD |
| | CBM | EDITSHM |
| | (human) | |
| SEQ ID NO: 17 | Lumican | MSLSAFTLFL ALIGGTSGQY |
| | | YDYDFPLSIY GQSSPNCAPE |
| | | CNCPESYPSA MYCDELKLKS |
| | | VPMVPPGIKY LYLRNNQIDH |
| | | IDEKAFENVT DLQWLILDHN |
| | | LLENSKIKGR VFSKLKQLKK |
| | | LHINHNNLTE SVGPLPKSLE |
| | | DLQLTHNKIT KLGSFEGLVN |
| | | LTFIHLQHNR LKEDAVSAAF |
| | | KGLKSLEYLD LSFNQIARLP |
| | | SGLPVSLLTL YLDNNKISNI |
| | | PDEYFKRFNA LQYLRLSHNE |
| | | LADSGIPGNS FNVSSLVELD |
| | | LSYNKLKNIP TVNENLENYY |
| | | LEVNQLEKFD IKSFCKILGP |
| | | LSYSKIKHLR LDGNRISETS |
| | | LPPDMYECLR VANEVTLN |
| SEQ ID NO: 18 | LAIR1 | MSLHPVILLV LVLCLGWKIN |
| | (murine) | TQEGSLPDIT IFPNSSLMIS |
| | | QGTFVTVVCS YSDKHDLYNM |
| | | VRLEKDGSTF MEKSTEPYKT |
| | | EDEFEIGPVN ETITGHYSCI |
| | | YSKGITWSER SKTLELKVIK |
| | | ENVIQTPAPG PTSDTSWLKT |
| | | YSIYIFTVVS VIFLLCLSAL |
| | | LFCFLRHRQK KQGLPNNKRQ |
| | | QQRPEERLNL ATNGLEMTPD |
| | | IVADDRLPED RWTETWTPVA |
| | | GDLQEVTYIQ LDHHSLTQRA |
| | | VGAVTSQSTD MAESSTYAAI |
| | | IRH |
| SEQ ID NO: 19 | LAIR1 | MSPHPTALLG LVLCLAQTIH |
| | (human) | TQEEDLPRPS ISAEPGTVIP |
| | | LGSHVTFVCR GPVGVQTFRL |
| | | ERESRSTYND TEDVSQASPS |
| | | ESEARFRIDS VSEGNAGPYR |
| | | CIYYKPPKWS EQSDYLELLV |
| | | KETSGGPDSP DTEPGSSAGP |
| | | TQRPSDNSHN EHAPASQGLK |
| | | AEHLYILIGV SVVFLFCLLL |
| | | LVLFCLHRQN QIKQGPPRSK |
| | | DEEQKPQQRP DLAVDVLERT |
| | | ADKATVNGLP EKDRETDTSA |
| | | LAAGSSQEVT YAQLDHWALT |
| | | QRTARAVSPQ STKPMAESIT |
| | | YAAVARH |
| SEQ ID NO: 20 | ECD LAIR1 | QEEDLPRPSI SAEPGTVIPL |
| | (human) | GSHVTFVCRG PVGVQTFRLE |
| | | RESRSTYNDT EDVSQASPSE |
| | | SEARFRIDSV SEGNAGPYRC |
| | | IYYKPPKWSE QSDYLELLVK |
| | | ETSGGPDSPD TEPGSSAGPT |
| | | QRPSDNSHNE HAPASQGLKA |
| | | EHLY |
| SEQ ID NO: 21 | LAIR2 | MSPHLTALLG LVLCLAQTIH |
| | (human) | TQEGALPRPS ISAEPGTVIS |
| | | PGSHVTFMCR GPVGVQTFRL |
| | | EREDRAKYKD SYNVFRLGPS |
| | | ESEARFHIDS VSEGNAGLYR |
| | | CLYYKPPGWS EHSDFLELLV |
| | | KESSGGPDSP DTEPGSSAGT |
| | | VPGTEASGFD AP |

In certain embodiments, the collagen-binding domain comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 7, or a portion thereof. In some embodiments, the collagen-binding domain is a variant comprising one or more amino acid substitutions, additions or deletions, optionally two, three, four, five, six, seven, eight, nine, ten or more amino acid substitutions, additions or deletions relative to the amino acid sequence of SEQ ID NO: 7. In some embodiments, the collagen-binding domain variant has increased binding affinity to collagen relative to a collagen binding affinity of the amino acid sequence of SEQ ID NO: 7. In some embodiments, the collagen-binding domain variant has decreased binding affinity to collagen relative to a collagen binding affinity of the amino acid sequence of SEQ ID NO: 7.

In certain embodiments, the collagen-binding domain comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 17, or a portion thereof. In some embodiments, the collagen-binding domain is a variant comprising one or more amino acid substitutions, additions or deletions, optionally two, three, four, five, six, seven, eight, nine, ten or more amino acid substitutions, additions or deletions relative to the amino acid sequence of SEQ ID NO: 17. In some embodiments, the collagen-binding domain variant has increased binding affinity to collagen relative to a collagen binding affinity of the amino acid sequence of SEQ ID NO: 17. In some embodiments, the collagen-binding domain variant has decreased binding affinity to collagen relative to a collagen binding affinity of the amino acid sequence of SEQ ID NO: 17.

In certain embodiments, the collagen-binding domain comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 18, or a portion thereof. In some embodiments, the collagen-binding domain is a variant comprising one or more amino acid substitutions, additions or deletions, optionally two, three, four, five, six, seven, eight, nine, ten or more amino acid substitutions, additions or deletions relative to the amino acid sequence of SEQ ID NO: 18. In some embodiments, the collagen-binding domain variant has increased binding affinity to collagen relative to a collagen binding affinity of the amino acid sequence of SEQ ID NO: 18. In some embodiments, the collagen-binding domain variant has decreased binding affinity to collagen relative to a collagen binding affinity of the amino acid sequence of SEQ ID NO: 18.

In certain embodiments, the collagen-binding domain comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 19, or a portion thereof. In some embodiments, the collagen-binding domain is a variant comprising one or more amino acid substitutions, additions or deletions, optionally two, three, four, five, six, seven, eight, nine, ten or more amino acid substitutions, additions or deletions relative to the amino acid sequence of SEQ ID NO: 19. In some embodiments, the collagen-binding domain variant has increased binding affinity to collagen relative to a collagen binding affinity of the amino acid sequence of SEQ ID NO: 19. In some embodiments, the collagen-binding domain variant has decreased binding affinity to collagen relative to a collagen binding affinity of the amino acid sequence of SEQ ID NO: 19.

In certain embodiments, the collagen-binding domain comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 20, or a portion thereof. In some embodiments, the collagen-binding domain is a variant comprising one or more amino acid substitutions, additions or deletions, optionally two, three, four, five, six, seven, eight, nine, ten or more amino acid substitutions, additions or deletions relative to the amino acid sequence of SEQ ID NO: 20. In some embodiments, the collagen-binding domain variant has increased binding affinity to collagen relative to a collagen binding affinity of the amino acid sequence of SEQ ID NO: 20. In some embodiments, the collagen-binding domain variant has decreased binding affinity to collagen relative to a collagen binding affinity of the amino acid sequence of SEQ ID NO: 20.

In certain embodiments, the collagen-binding domain comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 21, or a portion thereof. In some embodiments, the collagen-binding domain is a variant comprising one or more amino acid substitutions, additions or deletions, optionally two, three, four, five, six, seven, eight, nine, ten or more amino acid substitutions, additions or deletions relative to the amino acid sequence of SEQ ID NO: 21. In some embodiments, the collagen-binding domain variant has increased binding affinity to collagen relative to a collagen binding affinity of the amino acid sequence of SEQ ID NO: 21. In some embodiments, the collagen-binding domain variant has decreased binding affinity to collagen relative to a collagen binding affinity of the amino acid sequence of SEQ ID NO: 21.

Other exemplary tumor binding peptide sequences are provided below:

| SEQ | | |
|---|---|---|
| ID | | |
| NO: | Description | Sequence |
| 5 | Hyaluronic acid | GAHWQFNALTVR |
| | (HA) binding | |
| | peptide | |
| 6 | Fibronectin BP | TGLPVGVGYVVTVLT |
| 8 | Lyss polycation | KKKKK |
| 22 | integrin- | GCPRPRGDNPPLTCSQ |
| | binding | DSDCLAGCVCGPNGFC |
| | polypeptide | G |
| 23 | integrin- | GCPRPRGDNPPLTCKQ |
| | binding | DSDCLAGCVCGPNGFC |
| | polypeptide | G |

In certain embodiments, the tumor binding peptide comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 5, or a portion thereof. In some embodiments, the tumor binding peptide is a variant comprising one or more amino acid substitutions, additions or deletions, optionally two, three, four, five, six, seven, eight, nine, ten or more amino acid substitutions, additions or deletions relative to the amino acid sequence of SEQ ID NO: 5.

In certain embodiments, the tumor binding peptide comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 6, or a portion thereof. In some embodiments, the tumor binding peptide is a variant comprising one or more amino acid substitutions, additions or deletions, optionally two, three, four, five, six, seven, eight, nine, ten or more amino acid substitutions, additions or deletions relative to the amino acid sequence of SEQ ID NO: 6.

In certain embodiments, the tumor binding peptide comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 8, or a portion thereof. In some embodiments, the tumor binding peptide is a variant comprising one or more amino acid substitutions, additions or deletions, optionally two, three, four, five, six, seven, eight, nine, ten or more amino acid substitutions, additions or deletions relative to the amino acid sequence of SEQ ID NO: 8.

In certain embodiments, the tumor binding peptide comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 22 or SEQ ID NO: 23, or a portion thereof. In some embodiments, the tumor binding peptide is a variant comprising one or more amino acid substitutions, additions or deletions, optionally two, three, four, five, six, seven, eight, nine, ten or more amino acid substitutions, additions or deletions relative to the amino acid sequence of SEQ ID NO: 22 or SEQ ID NO: 23.

In some embodiments, the tumor binding peptide is at the N-terminus of the eGMCSF fusion polypeptide of the present technology. In a further embodiment, the tumor binding peptide is at the N-terminus of the eGMCSF fusion polypeptide and the mammalian GMCSF is at the C-terminus of the eGMCSF fusion polypeptide.

In other embodiments, the tumor binding peptide is at the C-terminus of the eGMCSF fusion polypeptide of the present technology. In a further embodiment, the tumor binding peptide is at the C-terminus of the eGMCSF fusion polypeptide and the mammalian GMCSF is at the N-terminus of the eGMCSF fusion polypeptide.

The eGMCSF fusion polypeptide of the present technology may further comprise a polypeptide tag (e.g., polyhistidine tag) and/or a heterologous protease cleavage site.

Methods for Making eGMCSF Fusion Polypeptides of the Present Technology

The eGMCSF fusion polypeptides of the present technology are made using recombinant DNA technology. In some aspects, the domains of the eGMCSF fusion polypeptides described herein (e.g., tumor binding peptides, GMCSF) are made in transformed host cells using recombinant DNA techniques. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The eGMCSF fusion polypeptides of the present technology are isolated and purified using one or more methods known in the art, including centrifugation, depth filtration, cell lysis, homogenization, freeze thawing, affinity purification, gel filtration, size exchange chromatography, ion exchange chromatography, hydrophobic interaction exchange chromatography, and mixed-mode chromatography. In certain embodiments, the fusion proteins described herein are purified by size exchange chromatography with a protein A resin. In certain embodiments, the fusion proteins described herein are purified by size exchange chromatography with Capto™Blue resin. In certain embodiments, the fusion proteins described herein are purified by size exchange chromatography with CaptureSelect™ HSA resin. In certain embodiments, the purified fusion proteins described herein are concentrated by any suitable method known in the art. In certain embodiments, the purified fusion protein is concentrated to a concentration of 0.1-100 mg/ml, 1-50 mg/ml, or 10-30 mg/ml. In certain embodiments, the purified fusion protein is concentrated to a concentration of 0.1-100 mg/ml, 1-50 mg/ml, or 10-30 mg/ml without detectable agreggation of the fusion protein. In certain embodiments, the purified fusion protein is concentrated to a concentration of about 20 mg/ml without detectable aggregation of the fusion protein.

### Therapeutic Methods of the Present Technology

In one aspect, the eGMCSF fusion polypeptides disclosed herein are useful for treating or preventing cancer in a subject in need thereof. Examples of cancer include, but are not limited to, melanoma, mesothelioma, pancreatic cancer, glioblastoma, breast cancer, ovarian cancer, lung cancer, colorectal cancer, or prostate cancer. A metastatic tumor can arise from a multitude of primary tumor types, including but not limited to those of prostate, colon, lung, breast and liver. Accordingly, the compositions used herein, comprising, e.g., eGMCSF fusion polypeptide, can be administered to a cancer patient.

In certain embodiments, the eGMCSF fusion polypeptides disclosed herein are used to treat cancer. In some embodiments, the eGMCSF fusion polypeptides disclosed herein are used to treat melanoma, mesothelioma, pancreatic cancer, glioblastoma, breast cancer, ovarian cancer, lung cancer, colorectal cancer, or prostate cancer.

Additionally or alternatively, in certain embodiments, the eGMCSF fusion polypeptides disclosed herein inhibit the growth and/or proliferation of tumor cells. In certain embodiments, the eGMCSF fusion polypeptides disclosed herein reduce tumor size. In certain embodiments, the eGMCSF fusion polypeptides disclosed herein inhibit metastases of a primary tumor. Additionally or alternatively, in certain embodiments, the eGMCSF fusion polypeptides disclosed herein reduce the incidence of and/or mitigate systemic immune-related adverse events (IRAEs).

In certain embodiments, administration of the eGMCSF fusion polypeptides disclosed herein to a subject do not result in cytokine release syndrome after administration to a subject. In certain embodiments, the subject does not experience grade 4 cytokine release syndrome. In certain embodiments, the subject does not experience one or more symptoms associated with grade 4 cytokine release syndrome selected from the group consisting of hypotension, organ toxicity, fever and/or respiratory distress resulting in a need for supplemental oxygen.

In another aspect, the eGMCSF fusion polypeptides disclosed herein are useful in methods for increasing responsiveness to immune checkpoint inhibitor therapy in a subject diagnosed with or suffering from cancer. Examples of immune checkpoint inhibitors include, but are not limited to, ipilimumab (Yervoy®; Bristol-Myers Squibb, Princeton, NJ), pembrolizumab (Keytruda®; Merck, Whitehouse Station, NJ), nivolumab (Opdivo®; Bristol-Myers Squibb, Princeton, NJ), atezolizumab (Tecentriq®; Genetech, San Francisco, CA), avelumab (Bavencio®; Merck, Whitehouse Station, NJ and Pfizer, New York, NY), and durvalumab (Imfinzi®; AstraZeneca, Cambridge, UK), pidilizumab (Curetech Ltd., Yavne, Israel), AMP-224 (GlaxoSmithKline, La Jolla, CA), MPDL3280A (Roche, Basel, Switzerland), MDX-1105 (Bristol Myer Squibb, Princeton, NJ), MEDI-4736 (Medimmune, Gaithersburg, MD), arelumab (Merck Serono, Darmstadt, Germany), tremelimumab (Pfizer, New York, NY), IMP321 (Immutep S. A., New South Wales, Australia), MGA271 (Macrogenics, Rockville, MD), BMS-986016 (Bristol-Meyers Squibb, Princeton, NJ), lirilumab (Bristol-Myers Squibb, Princeton, NJ), urelumab (Bristol-Meyers Squibb, Princeton, NJ), PF-05082566 (Pfizer, New York, NY), IPH2101 (Bristol-Myers Squibb, Princeton, NJ), MEDI-6469 (MedImmune, Gaithersburg, MD), CP-870,893 (Genentech, Oceanside, CA), Mogamulizumab (Kyowa Hakko Kirin, La Jolla, CA), Varlilumab (CelIDex Therapeutics, Hampton, NJ), Galiximab (Biogen Idec, Cambridge, MA), AMP-514 (Amplimmune, Gaithersburg, MD), AUNP 12 (Aurigene, Bangalore, India), Indoximod (NewLink Genetics, Ames, IA), NLG-919 (NewLink Genetics, Ames, IA), INCB024360 (Incyte, Wilmington, DE), or any combination thereof.

Determination of the Biological Effect of eGMCSF Fusion Polypeptides of the Present Technology

In various embodiments, suitable in vitro or in vivo assays are performed to determine the effect of a specific eGMCSF fusion polypeptide and whether its administration is indicated for treatment. In various embodiments, in vitro assays can be performed with representative animal models, to determine if a given eGMCSF fusion polypeptide exerts the desired effect on reducing or eliminating signs and/or symptoms of cancer. Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects. In some embodiments, in vitro or in vivo testing is directed to the biological function of one or more eGMCSF fusion polypeptides.

Animal models of cancer may be generated using techniques known in the art. Such models may be used to demonstrate the biological effect of eGMCSF fusion polypeptides in the prevention and treatment of conditions arising from disruption of a particular gene, and for determining what comprises a therapeutically effective amount of the one or more eGMCSF fusion polypeptides disclosed herein in a given context.

### Modes of Administration and Effective Dosages

Any method known to those in the art for contacting a cell, organ or tissue with one or more eGMCSF fusion polypeptides disclosed herein may be employed. Suitable methods include in vitro, ex vivo, or in vivo methods. In vivo methods typically include the administration of one or more eGMCSF fusion polypeptides to a mammal, suitably a human. When used in vivo for therapy, the one or more eGMCSF fusion polypeptides described herein are administered to the subject in effective amounts (i.e., amounts that have desired therapeutic effect). The dose and dosage regimen will depend upon the degree of the disease state of the subject, the characteristics of the particular eGMCSF fusion polypeptide used, e.g., its therapeutic index, and the subject's history.

The effective amount may be determined during pre-clinical trials and clinical trials by methods familiar to physicians and clinicians. An effective amount of one or more eGMCSF fusion polypeptides useful in the methods may be administered to a mammal in need thereof by any of a number of well-known methods for administering pharmaceutical compounds. The eGMCSF fusion polypeptides may be administered systemically or locally.

The one or more eGMCSF fusion polypeptides described herein can be incorporated into pharmaceutical compositions for administration, singly or in combination, to a subject for the treatment or prevention of cancer. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include intratumoral, parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more eGMCSF fusion polypeptides disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34 (7-8): 915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8): 915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4 (3): 201-9 (1994); and Gregoriadis, “Engineering Liposomes for Drug Delivery: Progress and Problems,” Trends Biotechnol., 13 (12): 527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more eGMCSF fusion polypeptides disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more eGMCSF fusion polypeptide concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more eGMCSF fusion polypeptides may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

For therapeutic and/or prophylactic applications, a composition comprising an eGMCSF fusion polypeptide disclosed herein, is administered to the subject. In some embodiments, the eGMCSF fusion polypeptide is administered one, two, three, four, or five times per day. In some embodiments, the eGMCSF fusion polypeptide is administered more than five times per day. Additionally or alternatively, in some embodiments, the eGMCSF fusion polypeptide is administered every day, every other day, every third day, every fourth day, every fifth day, or every sixth day. In some embodiments, the eGMCSF fusion polypeptide is administered weekly, bi-weekly, tri-weekly, or monthly. In some embodiments, the eGMCSF fusion polypeptide is administered for a period of one, two, three, four, or five weeks. In some embodiments, the eGMCSF fusion polypeptide is administered for six weeks or more. In some embodiments, the eGMCSF fusion polypeptide is administered for twelve weeks or more. In some embodiments, the eGMCSF fusion polypeptide is administered for a period of less than one year. In some embodiments, the eGMCSF fusion polypeptide is administered for a period of more than one year. In some embodiments, the eGMCSF fusion polypeptide is administered throughout the subject's life.

In some embodiments of the methods of the present technology, the eGMCSF fusion polypeptide is administered daily for 1 week or more. In some embodiments of the methods of the present technology, the eGMCSF fusion polypeptide is administered daily for 2 weeks or more. In some embodiments of the methods of the present technology, the eGMCSF fusion polypeptide is administered daily for 3 weeks or more. In some embodiments of the methods of the present technology, the eGMCSF fusion polypeptide is administered daily for 4 weeks or more. In some embodiments of the methods of the present technology, the eGMCSF fusion polypeptide is administered daily for 6 weeks or more. In some embodiments of the methods of the present technology, the eGMCSF fusion polypeptide is administered daily for 12 weeks or more. In some embodiments, the eGMCSF fusion polypeptide is administered daily throughout the subject's life.

### Combination Therapy

In some embodiments, one or more of the eGMCSF fusion polypeptides disclosed herein may be combined with one or more additional therapies for the prevention or treatment of cancer. Additional therapeutic agents include, but are not limited to, chemotherapeutic agents, immunotherapeutic agents (e.g., immune checkpoint inhibitors, see supra), radiotherapeutic agents etc.

In some embodiments, the one or more eGMCSF fusion polypeptides disclosed herein may be separately, sequentially or simultaneously administered with at least one additional therapeutic agent. Examples of additional therapeutic agents include, but are not limited to, alkylating agents, topoisomerase inhibitors, endoplasmic reticulum stress inducing agents, antimetabolites, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, phenphormin and targeted biological therapy agents (e.g., therapeutic peptides described in U.S. Pat. No. 6,306,832, WO 2012007137, WO 2005000889, WO 2010096603 etc.).

Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitor include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

Examples of immune checkpoint inhibitors include, but are not limited to, ipilimumab (Yervoy®; Bristol-Myers Squibb, Princeton, NJ), pembrolizumab (Keytruda®; Merck, Whitehouse Station, NJ), nivolumab (Opdivo®; Bristol-Myers Squibb, Princeton, NJ), atezolizumab (Tecentriq®; Genetech, San Francisco, CA), avelumab (Bavencio®; Merck, Whitehouse Station, NJ and Pfizer, New York, NY), and durvalumab (Imfinzi®; AstraZeneca, Cambridge, UK), pidilizumab (Curetech Ltd., Yavne, Israel), AMP-224 (GlaxoSmithKline, La Jolla, CA), MPDL3280A (Roche, Basel, Switzerland), MDX-1105 (Bristol Myer Squibb, Princeton, NJ), MEDI-4736 (Medimmune, Gaithersburg, MD), arelumab (Merck Serono, Darmstadt, Germany), tremelimumab (Pfizer, New York, NY), IMP321 (Immutep S. A., New South Wales, Australia), MGA271 (Macrogenics, Rockville, MD), BMS-986016 (Bristol-Meyers Squibb, Princeton, NJ), lirilumab (Bristol-Myers Squibb, Princeton, NJ), urelumab (Bristol-Meyers Squibb, Princeton, NJ), PF-05082566 (Pfizer, New York, NY), IPH2101 (Bristol-Myers Squibb, Princeton, NJ), MEDI-6469 (MedImmune, Gaithersburg, MD), CP-870,893 (Genentech, Oceanside, CA), Mogamulizumab (Kyowa Hakko Kirin, La Jolla, CA), Varlilumab (CelIDex Therapeutics, Hampton, NJ), Galiximab (Biogen Idec, Cambridge, MA), AMP-514 (Amplimmune, Gaithersburg, MD), AUNP 12 (Aurigene, Bangalore, India), Indoximod (NewLink Genetics, Ames, IA), NLG-919 (NewLink Genetics, Ames, IA), INCB024360 (Incyte, Wilmington, DE) or any combination thereof.

In certain embodiments, an additional therapeutic agent is administered to a subject in combination with the one or more engineered GMCSF (eGMCSF) fusion polypeptides disclosed herein such that a synergistic therapeutic effect is produced. For example, administration of one or more engineered GMCSF (eGMCSF) fusion polypeptides with one or more additional therapeutic agents for the prevention or treatment of cancer will have greater than additive effects in the prevention or treatment of the disease. For example, lower doses of one or more of the therapeutic agents may be used in treating or preventing cancer resulting in increased therapeutic efficacy and decreased side-effects. In some embodiments, the one or more engineered GMCSF (eGMCSF) fusion polypeptides disclosed herein are administered in combination with any of the at least one additional therapeutic agents described above, such that a synergistic effect in the prevention or treatment of cancer results.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure also provides kits for the prevention and/or treatment of cancer comprising one or more of any and all embodiments of the eGMCSF fusion polypeptides described herein. Optionally, the above described components of the kits of the present technology are packed in suitable containers and labeled for the prevention and/or treatment of cancer.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### EXAMPLES

The present technology is further illustrated by the following Examples, which should not be construed as limiting in any way.

### Example 1: Materials and Methods

We have engineered a novel recombinant GMCSF to improve intratumoral residence time and limit systemic immune-related adverse events (IRAEs), hereby referred to as engineered GMCSF (eGMCSF). The eGMCSF is fabricated by recombinantly fusing various tumor-binding peptides (TBPs) to the C-terminus of native murine GMCSF. To target HA, which is abundantly expressed in solid tumors, a novel 1.4 Da HA binding peptide (HABP) was chosen based on its specific binding to HA (K_{d}=1.65 μm) via hydrophobic interactions.¹⁶ The sequence of the fibronectin binding peptide (FnBP) is derived from the fibronectin binding component of bovine gelatin (K_{d}=77 nm).¹⁷ Similarly, the collagen binding motif (CBM) sequence was derived from human osteopontin protein (K_{d}=5 μm) and binds to collagen I through electrostatic and hydrophobic forces.¹⁸ Lastly, a five amino acid length polylysine motif (0.65 Da) was selected due to its known affinity towards charged HA via electrostatic interactions.¹⁹

Bacterial strains and plasmids. Escherichia coli (E. coli) BL21 (DE3) bacterial strain (NEB, USA) was used as the host strain for the bacterial transformation and subsequent engineered GMCSF (eGMCSF) protein expression. The amino acid sequence of the murine GMCSF is:

(SEQ ID NO: 9)
MAPTRSPITVTRPWKHVEAIKEALNLLDDMPVTLNEE
VEVVSNEFSFKKLTCVQTRLKIFEQGLRGNFTKLKGA
LNMTASYYQTYCPPTPETDCETQVTTYADFIDSLKTF
LTDIPFECKKPVQK.

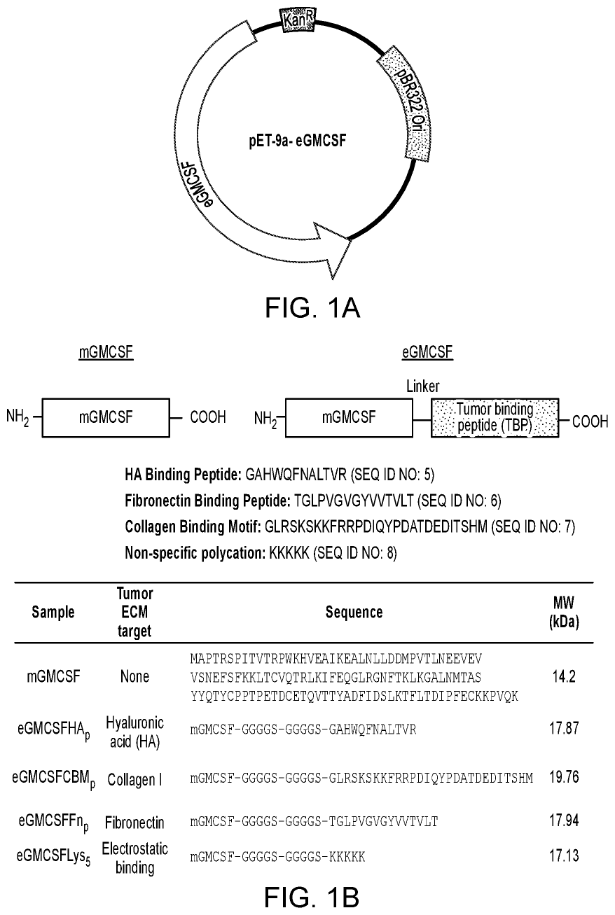

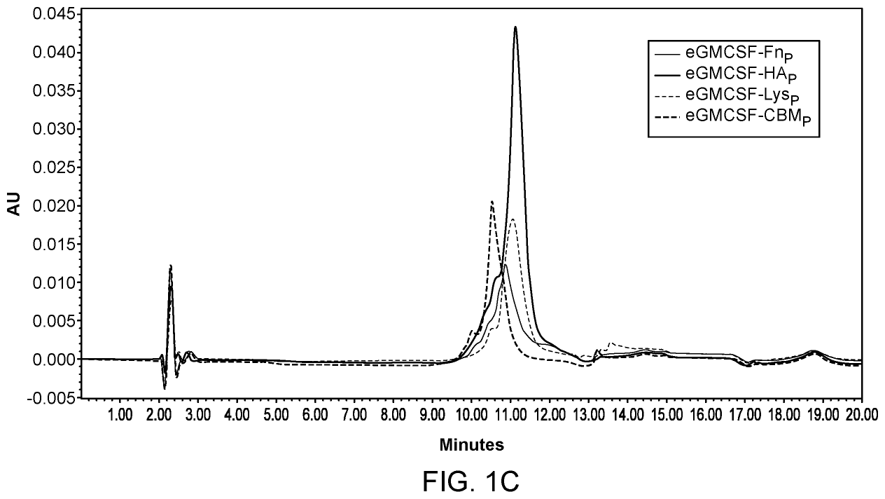

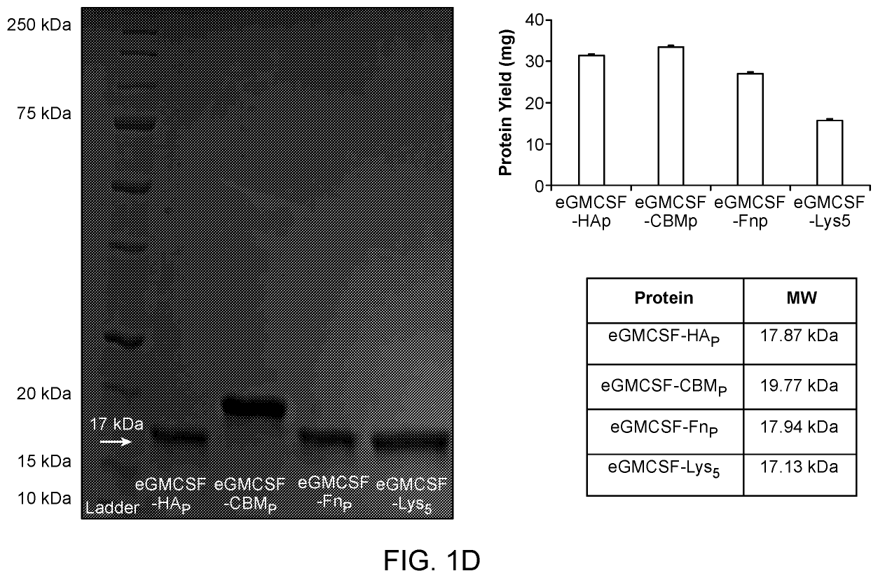

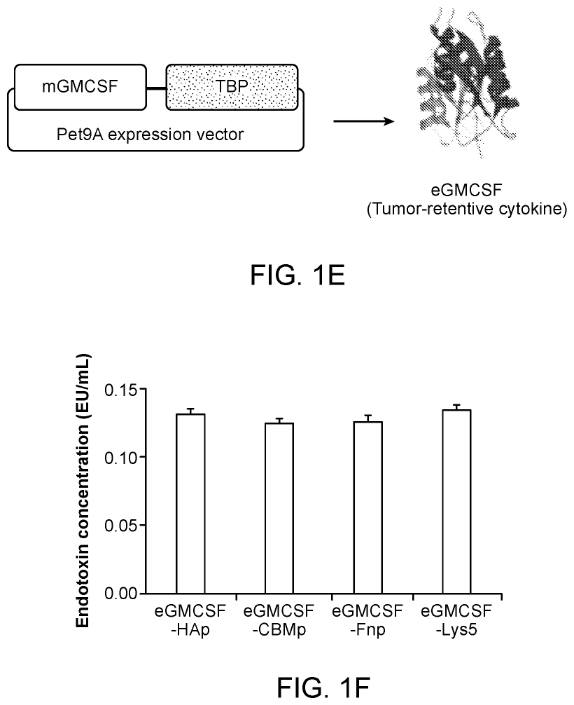

Three different eGMCSF variants were designed to bind to distinct tumor ECM components, such as HA, fibronectin, and collagen I (FIG. 1B). In addition, a variant of the protein that includes a non-specific polycation fused to GMCSF was generated to explore electrostatic binding of the engineered drug to the tumor tissue (eGMCSF-Lys5). The DNA sequence for the different eGMCSFs was cloned into a pET-9a expression vector between the XBa1 and BamH1 restriction sites by GenScript, USA. Specifically, each gene construct consists of a sequence encoding for a tumor binding motif introduced to the C-terminus of mGMCSF using a glycine linker [(GGGS)₂] (SEQ ID NO: 10) between them. Additionally, a polyhistidine tag (6×-His) (SEQ ID NO: 11) was incorporated via a TEV protease cleavage sequence ENLYFQ (SEQ ID NO: 12) at the N-terminus of the eGMCSF construct to aid in downstream protein purification (FIGS. 1A-1B, 1E). To enhance the soluble expression of eGMCSF in E. coli, co-expression of the recombinant protein was carried out with molecular chaperones in order to ensure correct protein folding as well as prevent any protease degradation during expression. A pkJE7 expression plasmid (Takara, Japan) encoding the dna K, dna J, and grp E chaperone was used for this purpose.

The amino acid sequences of the eGMCSF fusion polypeptides are provided below:

| | Tumor ECM | |
|---|---|---|
| Sample | target | Sequence |
| eGMCSF-HAₚ | Hyaluronic | MAPTRSPITVTRPWKHVEAI |
| | acid | KEALNLLDDMPVTLNEEVEV |
| | | VSNEFSFKKLTCVQTRLKIF |
| | | EQGLRGNFTKLKGALNMTAS |
| | | YYQTYCPPTPETDCETQVTT |
| | | YADFIDSLKTFLTDIPFECK |
| | | KPVQKGGGGSGGGGSGAHWQ |
| | | FNALTVR |
| | | (SEQ ID NO: 1) |
| eGMCSF-CBMₚ | Collagen I | MAPTRSPITVTRPWKHVEAI |
| | | KEALNLLDDMPVTLNEEVEV |
| | | VSNEFSFKKLTCVQTRLKIF |
| | | EQGLRGNFTKLKGALNMTAS |
| | | YYQTYCPPTPETDCETQVTT |
| | | YADFIDSLKTFLTDIPFECK |
| | | KPVQKGGGGSGGGGSGLRSK |
| | | SKKFRRPDIQYPDATDEDIT |
| | | SHM (SEQ ID NO: 2) |
| eGMCSF-Fnₚ | Fibronectin | MAPTRSPITVTRPWKHVEAI |
| | | KEALNLLDDMPVTLNEEVEV |
| | | VSNEFSFKKLTCVQTRLKIF |
| | | EQGLRGNFTKLKGALNMTAS |
| | | YYQTYCPPTPETDCETQVTT |
| | | YADFIDSLKTFLTDIPFECK |
| | | KPVQKGGGGSGGGGSTGLPV |
| | | GVGYVVTVLT |
| | | (SEQ ID NO: 3) |
| eGMCSF-Lys₅ | Electrostatic | MAPTRSPITVTRPWKHVEAI |
| | binding | KEALNLLDDMPVTLNEEVEV |
| | | VSNEFSFKKLTCVQTRLKIF |
| | | EQGLRGNFTKLKGALNMTAS |
| | | YYQTYCPPTPETDCETQVTT |
| | | YADFIDSLKTFLTDIPFECK |
| | | KPVQKGGGGSGGGGSKKKKK |
| | | (SEQ ID NO: 4) |

Soluble expression of eGMCSF in E. coli. The eGMCSF protein expression was performed by co-transforming the BL21 (DE3) cells with the pET-9a-eGMCSF and pKJE7 plasmids (dna K-dna J-grp E) in two steps. First, the host E. coli strain was transformed with a pKJE7 chaperone plasmid using the heat-shock technique at 42° C., followed by the second round of transformation with the pET-9a-eGMCSF plasmid. The co-transformed cells were then grown on Luria-Bertani (LB) agar plates containing the antibiotics, Kanamycin (25 μg/ml), and Chloramphenicol (34 μg/ml). The presence of antibiotic resistance genes on the respective plasmids ensures that only the successfully co-transformed cells would grow on the LB agar plates. Consequentially, the positive colonies were selected and inoculated in a LB broth culture supplemented with antibiotics then incubated overnight at 37° C. and 180 rpm. The next day, the bacterial culture was sub-cultured by inoculating 1% of the overnight culture into fresh LB media (+Antibiotics) containing 0.5 mg/ml arabinose and incubated at 37° C. and 250 rpm until the bacterial cells reached exponential phase (OD₆₀₀=0.6-0.8). This step initiates the expression of the chaperone proteins. Subsequently, the expression of eGMCSF was induced by the addition of 0.5 mM isopropyl b-D-1-thiogalatopyranoside (IPTG) and incubation at 30° C. and 250 rpm for 4 h.

In order to harvest the protein of interest, the bacterial cultures were centrifuged at 4000 rpm for 40 mins at 4° C. Cells were then suspended in a Lysis buffer (10 mM Tris-HCl, 30 mM NaCl, pH 7.5) and mechanically disrupted using a sonicator (85% intensity, 4 mins, Pulse mode—10 s on, 20 s off). Soluble and insoluble protein fractions were separated by centrifugation at 10,000 g for 20 mins. The insoluble fraction was then resuspended in a refolding buffer (6M GuHCl (Guanidinium chloride), 50 mM Tris-HCl, 20 mM β-mercaptoethanol, (pH 8) and incubated for 15 mins at room temperature. At this point, the soluble protein was extracted by centrifugation at 10,000 g for 20 mins and filtered using a 0.8 μm filter.

Finally, the soluble protein was purified by performing immobilized metal affinity chromatography (IMAC) purification using a Ni-NTA affinity column. The presence of the 6×-His tag in the eGMCSF sequence allows the tagged protein to bind to the Ni-NTA column with high affinity while the untagged proteins are removed by rinsing the column with a wash buffer (6M GuHCl, 10 mM Imidazole, 10 mM Tris-HCl, pH 6.9). The target protein was then eluted using an elution buffer (6M GuHCl, 250 mM Imidazole, 10 mM Tris-HCl, pH 5.9). Finally, GuHCl was removed from the protein sample by dialyzing (MWCO: 3500 Da) against 10% acetic acid at 4° C. for 24 h (R. Malekian, S. Sima, A. Jahanian-Najafabadi, F. Moazen and V. Akbari, Protein Expr Purif, 2019, 160, 66-72).

Analytical methods. The concentration of the purified protein was quantified by estimating the absorbance at 280 nm using a NanoDrop spectrophotometer. The amount of endotoxin present in the protein product was identified using a Limulus amebocyte lysate (LAL) assay (GenScript, USA) and compared to commercially available murine GMCSF (Peprotech, USA). Since eGMCSF was synthesized in a gram-negative bacterium (E. coli), it is essential to measure the concentration of bacterial endotoxin in the final protein product as endotoxins are pyrogenic in nature and can cause adverse reactions in humans and mice alike.

Furthermore, the protein samples were analyzed by 12% sodium dodecyl sulfate-polyacrylamide gel electrophoresis (SDS-PAGE) intended for confirming the molecular weight (MW) and purity of the different eGMCSFs. The purity of the expressed protein product was further confirmed by RP-HPLC (Reverse Phase High Performance Liquid Chromatography) using a C4 column (3.5 μm, 4.6×150 mm, Waters, USA) and purified with a linear gradient of 30-60% of buffer B (Acetonitrile, 0.05% trifluoroacetic acid) over buffer A (H₂O, 0.05% trifluoroacetic acid) over 20 mins with a flow rate at 1 mL/min and detection at 280 nm.

Biophysical characterization. Considering the molecular conformation of a protein determines its biological activity, it is imperative to confirm that the expressed proteins are correctly folded. The three-dimensional conformation of the recombinant eGMCSF variants were analyzed by circular dichroism (CD) using a J-1500 spectrophotometer (Jasco, USA). The CD Spectra were collected at 22° C. with a pathlength of 0.1 cm. The far-UV region (190-250 nm) CD profile of expressed eGMCSF variants were compared to that of native mGMCSF (Peprotech, USA). All samples consisted of 0.2 mg/mL of protein suspended in distilled water (DI H2O). A CD spectrum displaying a positive peak at 193 nm and negative peaks at 222 nm and 208 nm is indicative of an α-helical structure. BestSel (Beta Structure Selection) online tool was used to quantify the percentage of α-helices in each protein structure (K. L. Maxwell, D. Bona, C. Liu, C. H. Arrowsmith and A. M. Edwards, Protein Sci, 2003, 12, 2073-2080).

Furthermore, the particle size (hydrodynamic diameter) of the different eGMCSF proteins was evaluated using dynamic light scattering (DLS, Wyatt Technology). For the DLS measurements, all samples were prepared in 1×PBS (phosphate buffered saline, pH 7.4) and analysis was carried out at room temperature. mGMCSF was used as a control.

In vitro dendritic cell (DC) Proliferation assays. JAWSII DCs (ATCC, USA) were cultured in Alpha-MEM media supplemented with 20% fetal bovine serum (FBS), and 1% penicillin/streptomycin (P/S) at 37° C. and 5% CO₂ for approximately one week prior to studies. For the proliferation assay, cells were seeded in a 96 well plate at a seeding density of 10,000 cells/well and supplemented with the various treatment groups (Table A) followed by incubation at 37° C. and 5% CO₂ (n=4).

TABLE A

| Groups | Protein concentration |
|---|---|
| Control | No treatment |
| mGMCSF | 1, 3, 5 ng/mL |
| eGMCSF-HAₚ | 1, 3, 5 ng/mL |
| eGMCSF-CBMₚ | 1, 3, 5 ng/mL |
| eGMCSF-Fnₚ | 1, 3, 5 ng/mL |
| eGMCSF-Lys₅ | 1, 3, 5 ng/mL |

After 5 days, 75 μmol/L of cell proliferation reagent-Resazurin (7-hydroxy-3H-phenoxazin-3-one 10-oxide) was added to each well and incubated for 4 h at 37° C. The corresponding fluorescence intensity was measured using a fluorescence microplate reader (BioTek, USA) at excitation 560 nm, emission 590 nm. An increase in fluorescence intensity is proportional to the number of viable cells as the resazurin (non-fluorescent) is reduced by cellular enzymes to form the resorufin (fluorescent). Cells without any protein treatment served as control.

In vitro dendritic cells (DC) maturation assay. For the in vitro maturation studies, JAWSII DCs were seeded in a 48 well plate (30,000 cells/well) in complete fresh medium supplemented with the various treatment regimens (Table A). Cells without any treatment served as a control. To confirm the maturation of the DCs, cells were harvested at 0 h and 48 h and labeled with PE anti-mouse CD86, FITC anti-mouse CD80, and Pacific Blue™ anti-mouse CD40 antibodies (BioLegend, USA). Simultaneously, all the groups were stained with the 7-AAD Viability Staining Solution (BioLegend, USA) to exclude dead cells from the sample set (n=4). The multicolor flow cytometry acquisition and analysis was achieved with a BD FACS Fusion cytometer (M. Yong, D. Mitchell, A. Caudron, I. Toth and C. Olive, Vaccine, 2009, 27, 3313-3318).

Fluorescent labeling of eGMCSF variants. The individual proteins were labeled with a fluorescent dye-fluorescein isothiocyanate (FITC). 1 equivalent of eGMCSF protein (50 μmol) was mixed with 5 equivalents of FITC (250 μmol). Freshly prepared 50 mM carbonate buffer was added to the reaction and the mixture was stirred for 1 hour in the dark at room temperature (K. D. Apley, J. D. Griffin, S. N. Johnson, C. J. Berkland and B. J. DeKosky, J Vis Exp, 2020, DOI: 10.3791/61827). To remove the free dye, form the fluorophore conjugated samples, dialysis was performed using a 7000 kDa molecular weight cutoff dialysis tubing in a stirred 5 L bucket with distilled water in the dark at room temperature. Samples were dialyzed for 24 hours with the dialysis solution being replaced every 6 hours. The degree of dye labeling was performed by UV-Vis spectroscopy. The absorbance conjugated samples at 280 nm and 490 nm (peak absorption wavelength of dye) were recorded. The following equation was used to calculate the degree of dye-labeling:

Molar dye/protein=(A₄₉₀/ε_{dye})*{εₚᵣₒₜₑᵢₙ/[A₂₈₀−(CF*A₄₉₀)]}

Where, CF is the correction factor due to absorbance of FITC at 280 nm, ε_{dye} and εₚᵣₒₜₑᵢₙ are the molar extinction coefficients of the dye and protein, respectively. A₄₉₀ and A₂₈₀ are the absorbance of the conjugated samples at 280 and 490 nm, respectively.

The degree of labeling was calculated as F/P molar ratio, which is the ratio of moles of FITC to moles of protein. The F/P molar ratio for the different proteins was as follows-eGMCSF-HAₚ (0.33), eGMCSF-CBMₚ (0.32), eGMCSF-Fnₚ (0.31), eGMCSF-Lys (0.55), and mGMCSF (0.52). Since the degree of labeling for each protein is slightly different due to varied protein sequences, all the RFUs (relative fluorescence units) were standardized to the respective standard curves to make the data directly comparable.

Intratumoral delivery simulation. The tumor binding affinity of eGMCSF-HAₚ, eGMCSF-Fnₚ, eGMCSF-CBMₚ, and eGMCSF-Lys₅ to different tumor ECM components like HA, fibronectin, and collagen I were evaluated using a dispersed gel model.

eGMCSF-HAₚ and eGMCSF-Lys₅: The binding efficacy of eGMCSF-HAₚ to HA was assessed by measuring the retention within a highly viscous HA gel. 35 mg/ml of 1.5 MDa HA (Lifecore Biomedical) was mixed with an eGMCSF-HAₚ-FITC solution (1 μg/mL) and left to dissolve overnight on an end-over-end rotator. This ensures the 3D incorporation of the eGMCSF-HAₚ within the gel matrix. The gel mixture was then weighed out into a 96 well plate at 150 mg/well and the plate was centrifuged to remove bubbles. Finally, each well was topped with 150 μL of 1× phosphate-buffered saline (PBS) and left on a shaker at 250 rpm at 4° C. FITC labeled-unmodified mGMCSF was incorporated into HA gels served as control. The release supernatants were collected at various time points (0, 1, 2, 3, 6, 12, 24 h). The amount of mGMCSF or eGMCSF-HAₚ released from the HA gels was quantitively evaluated by carrying out fluorescent spectroscopy on the release supernatants at excitation 490 nm and emission 525 nm. All the RFUs (relative fluorescent intensity) were normalized to the individual standard curves to get the cumulative protein released (pmole).

The binding efficacy of positively charged eGMCSF-Lys₅ to HA which is inherently negatively charged was tested as detailed above. Electrostatic interactions should facilitate increased retention of eGMCSF-Lys₅ within the dispersed gel model.

eGMCSF-Fnₚ and eGMCSF-CBMₚ: The retention of eGMCSF-Fnₚ was evaluated in a dispersed gel consisting of 35 mg/mL of HA (1.5 MDa) and plasma fibronectin (0.25 mg/mL) whereas, eGMCSF-CBMₚ was evaluated in a gel made of 35 mg/mL of HA (1.5 MDa) and 1 mg/mL Collagen I. The in vitro tumor binding studies were performed in an identical method to the detailed above.

Competitive inhibition. To determine the driving force behind the retention of eGMCSF within the dispersed gel matrix, the binding was competitively inhibited by the corresponding tumor binding peptide (TBP). The study was conducted by dissolving the tumor ECM components for the individual dispersed gels in 0.5 mL of corresponding TBP (varying concentration based on K_{d}, dissociation constant of peptide). The gel mixture was left on an end-to-end rotator until fully incorporated (˜6 hours). Next, 0.5 mL of FITC-conjugated eGMCSF variant (1 μg/mL) was added to the above mixture and left on end-to end rotator until fully incorporated (1-2 hours). Release supernatants were collected, and quantitative analysis was performed to measure protein release (Ex 490 nm, Em 525 nm).

Ex vivo tumor binding study. All animal experiments were carried out at the University of Kansas (KU) Animal Care Unit, in compliance with the “Guide for the Care and Use of Laboratory Animals” and accredited by the Association for the Assessment and Accreditation of Laboratory Animal Care International (AAALAC). All studies were done in compliance to a protocol approved by the IACUC committee at KU. Orthotopic tumors were generated by subcutaneously injecting 1×10⁶ AT84 oral squamous cell carcinoma (Head and neck cancer; HNSCC) into the neck of wildtype C3H mice (6-8 weeks old, 20-25 g, Male). The tumors were resected when they reached ≈900 mm³, (approximately 30 days after tumor induction). Tumor volume was monitored every third day using vernier calipers and calculated using the following equation:

Tumor volume (mm³)=0.52×(Width)²×Length

Fifty microliters of FITC-labeled GMCSF (200 ng in 4% Mannitol) was injected into individual tumors using insulin syringes. This IT injection is analogous to the clinical dosing regimen proposed in various in vivo GMCSF cytokine therapies. After injection, the tumors were transferred to a conical centrifuge tube containing 2 mL of physiological buffer (1×PBS). Tumors injected with FITC-labeled unmodified mGMCSF and 4% Mannitol served as negative and vehicle controls, respectively. Release supernatants were collected every three hours for the first 24 hours, and then every 3 days for the duration of 22 days. At each time point, the entire volume of supernatant was removed and replaced with 2 mL of fresh PBS. The amount of mGMCSF or eGMCSF released from the resected tumors was assessed by carrying out fluorescent spectroscopy as detailed in the sections above. Fluorescent imaging of the tumors was done using the MaestroFlex imager (Cambridge Research and Instrumentation, MA) to visualize the eGMCSF persistence at the end of the study (Day 23). Individual images were processed using Image J by normalizing each tumor's fluorescent intensity to that of the vehicle control to negate tissue autofluorescence.³⁷

In vivo therapeutic efficacy. Murine colon cancer models were generated by anesthetizing BALB/c mice (5% isoflurane in O₂, 5 mins) and injecting 1×10⁵ CT26 cells in 50 μL of Matrigel (Catalog no, Corning, USA) into the hind leg of mice (50:50 sex ratio). Drug treatments began when tumors reached around 100-200 mm³ in size, generally days 10-12 days after cell injection. To establish an ideal dosage a safety study was conducted by injecting mice with 3 different doses of eGMCSF (10 μg, 40 μg and 80 μg) in 4% mannitol (50 μL).^{13, 38} Mice injected with 50 μL of 4% mannitol served as vehicle controls. Drugs were administered every 3 days for a total of 5 drug injections. The tumor progression was monitored twice a week as reported above.³⁹

Upon establishing the minimum effective dose (MED) from the study above, the therapeutic efficacy of eGMCSF variants was evaluated by conducting the study as a monotherapy and as a combination therapy with mouse anti-PD1 checkpoint inhibitor (BioXcell, USA) using the colon cancer mice model. All mice received eGMCSF injections as detailed in the safety study and were compared to mice receiving unmodified mGMCSF. For the combination therapy, 250 μg of anti-PD1 was administered intraperitoneally (i.p injection) once a week for the entire duration of the study. Tumor size was be determined every third day and blood samples were collected two hours after the 1^{st} and 5^{th} treatment, to quantify inflammatory cytokines associated with systemic toxicity. At the end of the study, the mice were euthanized and the tumors were harvested and cut in half. One half was frozen in OCT media (Fisher Scientific) for immunohistochemistry (IHC) analysis while the other half was cut into small pieces (<5 mm) and stored in RNA Later solution (Ambion, Inc. Austin, TX) for RNA sequencing.

Immunohistochemistry staining. Upon tumor harvesting, the tumors halves stored in the OCT media (Fischer scientific, USA) were cryosectioned using a Cryotome instrument (ThermoFischer, USA) to obtain 6 μm thick tissue sections. The tumor samples were fixed in 10% formaldehyde and blocked with 10% goat serum (1×PBS). The slides were stained with 5 μg/mL of primary antibodies (BioLegend: Alexa Flour 400 anti-mouse CD8a, Alexa Flour 594 anti-mouse CD11b, Alexa Flour 647 anti-mouse CD11c) and incubated overnight at 4° C. The samples were then counterstained with Hoechst 33342 to visualize the nuclei and lastly mounted using the Fluoromount-G™ slide mounting medium (SouthernBioTech, USA). All images were obtained using an Olympus IX-81 inverted epifluorescence microscope (10× magnification) and the acquired images were processed using Slidebook 6.0.⁴⁰

Statistical analysis. All experimental data were calculated as mean±SD (standard deviation). The variation among the test groups was quantified using the paired two-tailed Student's t-test and one-way ANOVA for repeated measurements with Tukey post hoc comparisons. Data analysis for the animal studies involved 2-way ANOVA with Sidak's multiple comparisons test. Statistical significance for all data sets was set as p<0.05 (GraphPad Prism Software). (*=p<0.05, **=p<0.01, and ***=p<0.001.)

### Example 2: Soluble Expression of eGMCSE in E. coli

To develop effective IT cytokine immunotherapies, the eGMCSF variants detailed in FIG. 1B were expressed utilizing the pET-9a vector within E. coli expression systems.^{41, 42} Notably, the different eGMCSFs variants were synthesized in separate bacterial cultures for optimal downstream processing and purification. Overall, this methodology resulted in high protein yields of 3.75 mg-8.75 mg per 1 L of culture for the various eGMCSF variants as quantified by UV-Vis spectroscopy (A₂₈₀) (FIG. 1D).²⁷ Purified eGMCSF proteins were analyzed using SDS-PAGE, which separates proteins based on their molecular weights. The presence of distinct bands at the corresponding 17 kDa and 19 kDa MW markers in FIG. 1D confirmed the expression of the desired eGMCSF variants. The amount of endotoxin present in the protein product was and compared to commercially available mGMCSF (Peprotech, USA). The recombinant protein products had endotoxin levels ranging from 0.124-0.134 EU/mL, which is well within a safe and acceptable range considering commercial mGMCSF contains an endotoxin value of 1 EU/mL (FIG. 1F).³²

The purity of the expressed protein product was further confirmed by RP-HPLC (Reverse Phase High-Performance Liquid Chromatography). The presence of a single peak on the corresponding chromatographs further reiterated the synthesis of the engineered proteins with no undesired by-products in the final formulation. Interestingly, all four eGMCSF variants showed very similar retention times within the HPLC column, indicating comparable physiochemical properties (FIG. 1C). Due to similar molecular characteristics all the synthesized proteins will effectively display similar transport properties and can be characterized accordingly. The slight changes in retention time between the variants may be attributed at least in part to the presence of the different peptide tags in their structure. For instance, we observe a slight up-field shift in peak with the eGMCSF-Lys₅ variant due to the presence of a charged polylysine tag. Overall, we have devised a highly efficient methodology for synthesizing different tumor binding cytokine therapies-eGMCSF-HAₚ, eGMCSF-CBMₚ, eGMCSF-Fnₚ, and eGMCSF-Lys₅ that result in purified protein products with high yield.

### Example 3: Biophysical Characterization of eGMCSE

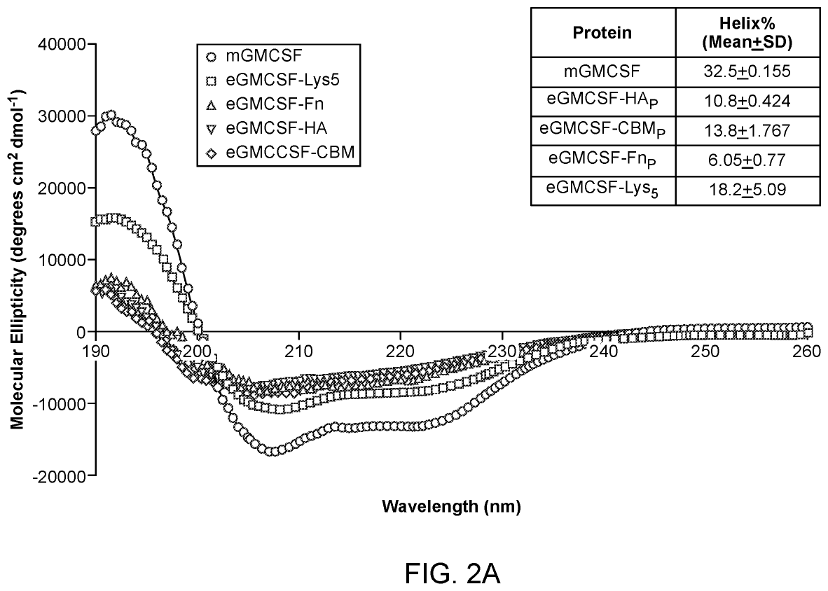

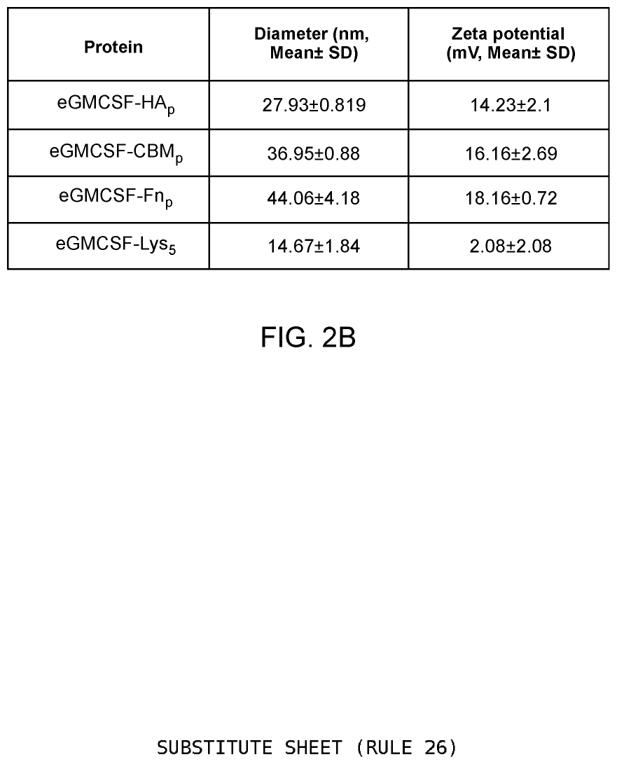

The secondary structure of the recombinant eGMCSF was evaluated by performing circular dichroism (CD) spectroscopy. The CD spectra of the recombinant proteins were similar to that of unmodified commercial mGMCSF (Peprotech, USA), which has four α-helices.⁴³ The CD curves for all proteins showed a positive peak at 193 nm and two negative peaks at 208 nm and 222 nm, which is characteristic of an α-helical rich structure (FIG. 2A).^{44, 45} We reasoned that the reduction in α-helical character in the eGMCSF variants was due to the presence of the different tumor binding peptides (TBPs) in its structure, which displays a random coil conformation (FIG. 11). Based on these results, we concluded that despite the C-terminus addition of the TBPs, the molecular integrity of the eGMCSF proteins remains predominantly unaffected. FIG. 2B shows Dynamic light scattering (DLS) of eGMCSF variants of the present technology.

For IT therapies, high-affinity molecules may exhibit poor penetration into the tumor mass due to a phenomenon called binding site barrier effect. Conversely, after administration, low affinity molecules may leach out from the tumor tissue rapidly. Therefore, the TBPs in the eGMCSF compounds were specifically selected to have an intermediate binding affinity in an attempt to avoid the binding site barrier effect and achieve a homogenous drug distribution within the tumor tissue.^{46, 47} Interestingly, if any eGMCSF were to leach out from the injection site either due to the K_{d} of the TBPs or because of ECM remodeling, these drugs may be preferentially trafficked to the draining lymph nodes.^{48, 49} Consequently, in addition to immune stimulation in the primary tumors, eGMCSF may further prime DCs and resident T cells in the lymph node, which may drive peripheral immunity against cancer (abscopal effect).^{50, 51}

### Example 4: Immunomodulatory Effects of eGMCSF

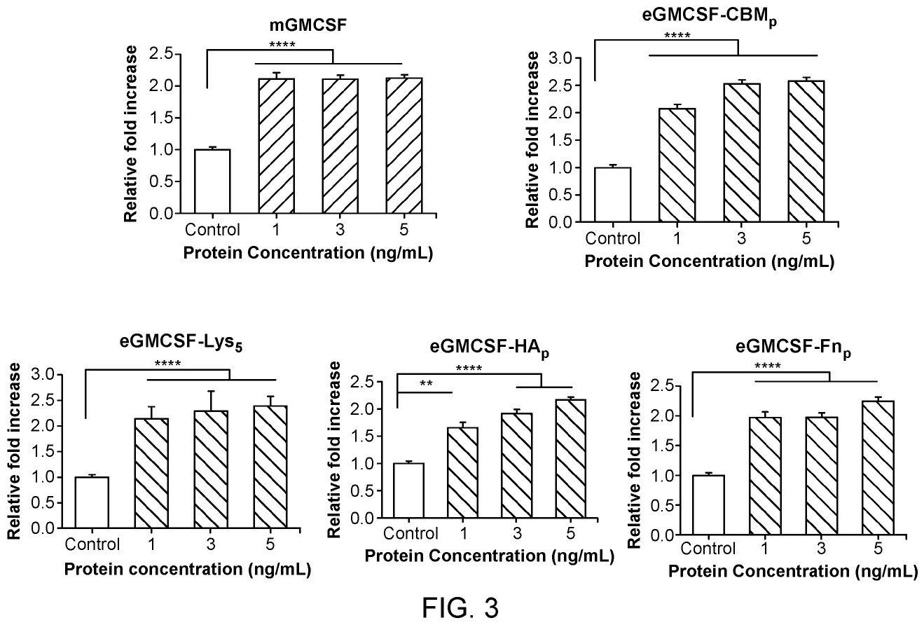

One of the immunological functions of GMCSF is to induce the recruitment and subsequent maturation of DCs that are essential for antigen capture and presentation. Upon maturation, DCs migrate from tumor tissue (site of antigen capture) to lymphoid organs and present the tumor-specific antigen via surface MHC (major histocompatibility complex) to naïve T cells. This consequently initiates a tumor-specific immune response that promotes the primed CD8⁺ and CD4⁺ T cells to infiltrate tumor tissue.^{43, 52} To elucidate the immunological activity of eGMCSF, we evaluated its effect on the proliferation of murine DCs (JAWS II cells). A resazurin assay was used to calculate the cellular proliferation as a measure of metabolic activity. Intracellular enzymes reduce the resazurin dye to produce the fluorescent product, resorufin and the fluorescent signal's intensity is commensurate with the number of viable cells. All four eGMCSF treatments resulted in a 1.5-2.5 fold concentration-dependent increase in cell proliferation in contrast to the control (FIG. 3). This effect was comparable to the cytokine activity of unmodified commercial mGMCSF, which showed a similar two-fold increase in DC proliferation. We concluded that despite the recombinant modification, the potency of the recombinant eGMCSFs remained roughly equivalent to that of native mGMCSF.

### Example 5: Intratumoral Retention of eGMCSF within Tumor Models

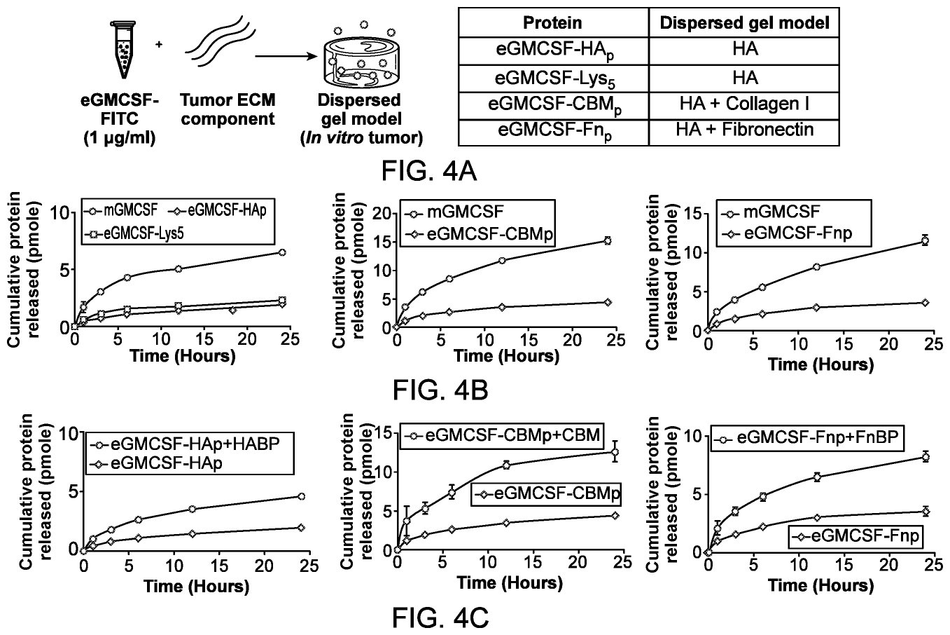

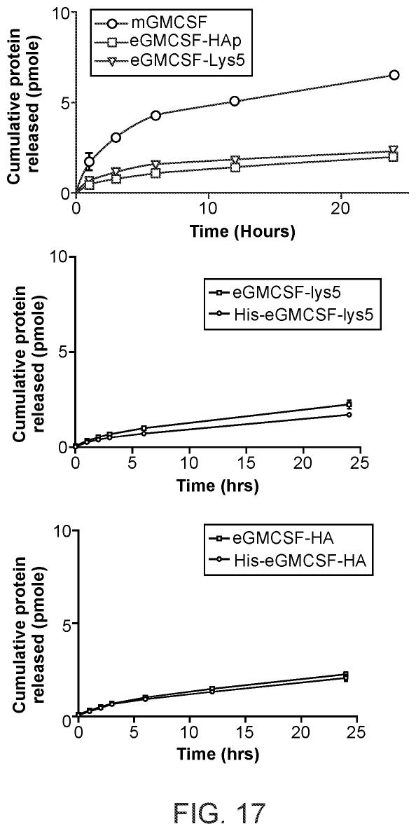

To study the tumor ECM binding capability of the eGMCSF proteins, their retention within a dispersed gel model was monitored. FIG. 4A depicts the workflow for fabricating the dispersed gel model, which has been used to simulate IT injection.⁵³ The eGMCSF-HAₚ and eGMCSF-Lys₅ proteins were studied using a highly viscous HA-based gel, whereas the binding of eGMCSF-CBMₚ and eGMCSF-Fnₚ was explored in HA gels supplemented with collagen I and fibronectin, respectively. Since HA is the main component of TME, it was used as the base material in each IT delivery simulation to mimic the mechanical and transport properties of tumor ECM.^{21,53} The retention/immobilization of eGMCSF variants with HIS-tag in hyaluronic acid gels was similar to eGMCSF variants without the HIS-tag (FIG. 17).

Over the course of 25 hours, we observed a more significant retention of the eGMCSF variants within their respective gel models. In contrast, the unmodified GMCSF was released from the gel model at a much faster rate (FIG. 4B). Furthermore, a competitive inhibition study was conducted to deduce the mechanism of action of these engineered IT proteins. In this case, the free TBP was first added to the gel model to occupy and saturate the available binding sites, followed by the addition of eGMCSF-HAₚ, eGMCSF-CBMₚ, and eGMCSF-Fnₚ to the respective tumor models. The TBPs (HABP, CBM, and FnBP) competitively inhibited the binding of eGMCSF to the ECM molecules leading to greater release of drug from the gel model (FIG. 4C). This confirmed that the driving force behind the binding efficacy of eGMCSF is the presence of the TBP in its structure. The concentrations of the TBPs were chosen based on their dissociation constant, K_{d}, which is the concentration at which half the available binding sites were occupied. (K_{d}: HABP-1.65 μM, CBM-5 μM, FnBP-77 nM).^{17, 54, 55} Due to the small size of the polylysine tag and ample availability of negatively charged HA, free polycation peptide could not competitively inhibit the binding of eGMCSF-Lys₅ and therefore was not investigated in this case.

Since the IT delivery simulation detailed above is rather simplistic in its composition and restricts diffusion in a single radial direction, the drug retention was further evaluated within ex vivo tumor models to comprehensively study the potency of eGMCSF as an IT drug. Orthotopic HNSCC tumors were generated by inoculating mice with AT84 cells and resecting them once they reached an average volume of 900 mm³ (30 days post tumor induction) (FIG. 5A). The resected tumors were measured and distributed between the different test groups to ensure a negligible difference in average tumor volume between the groups (FIG. 5C). The different FITC-labeled drugs were intratumorally injected (50 μL, in 4% Mannitol) into the ex vivo tumors, and the drug released into the surrounding physiological buffer was calculated (FIG. 5B). The release profile of the eGMCSF variants and mGMCSF is shown in FIG. 5D. To negate tissue autofluorescence, the average signal of the vehicle group was subtracted from each test group. Since HNSCC tumors are rich in HA, collagen I, and fibronectin, all the four eGMCSF proteins displayed increased IT residence time in contrast to unmodified mGMCSF, which leached out from the tumor tissue at a faster rate. The binding performance of the engineered drugs were as follows eGMCSF-HAₚ>eGMCSF-Fnₚ>eGMCSF-CBMₚ>eGMCSF-Lys. Without wishing to be bound by theory, this trend may be because HA has overlapping binding sites that allow for more than one eGMCSF-HAₚ to bind to a single HA molecule.⁵⁵ Correspondingly, the FnBP present in eGMCSF-Fnₚ is known to bind to both fibronectin and collagen-1, rendering it with better tumor binding potential.¹⁷ Lastly, eGMCSF-Lys₅ has the lowest binding efficacy of the engineered cytokines perhaps due to the incorporation of the shortest tumor binding domain compared to the other eGMCSFs. Therefore, the protein engineering strategy detailed in this research has successfully altered the drug properties to generate tumor-retentive cytokinesv.⁵⁶

### Example 6: In Vivo Therapeutic Efficacy of eGMCSF in Murine Colon Cancer Model

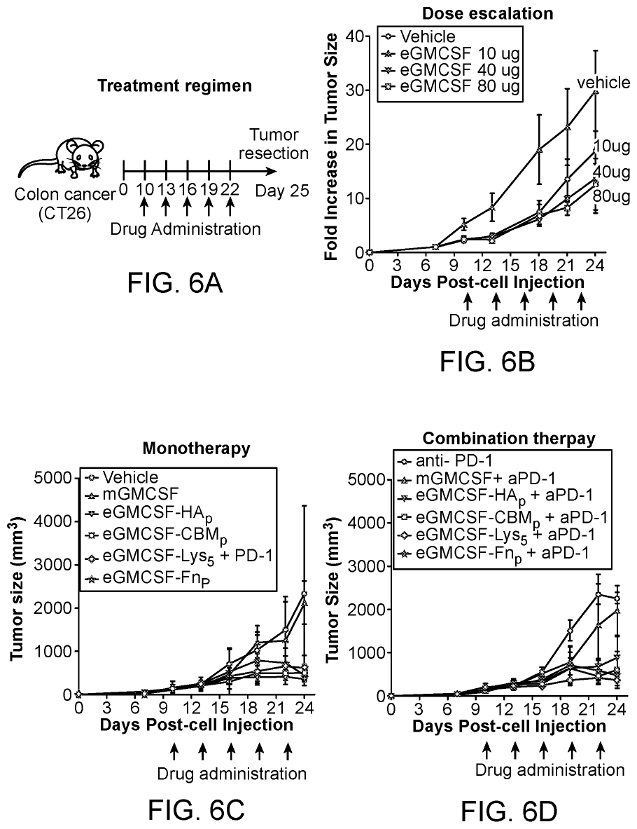

To establish a safe working dose of the engineered drug, a dose-escalation study was performed in diseased mice models using the eGMCSF-HAₚ variant. BALB/c mice expressing colon cancer tumors (CT26) received IT injections of eGMCSF-HAₚ twice weekly for a total of 5 injections (FIG. 6A). Previously reported doses for the IT treatment of melanoma with GMCSF used a low (10 μg), medium (40 μg), and high dose (80 μg) of the drug.^{38, 57} Mice receiving 50 μL of placebo injections (4% mannitol) acted as the vehicle control. FIG. 6B shows that the treatment with eGMCSF-HAₚ at doses 40 μg and 80 μg effectively reduced tumor progression in comparison to the vehicle control. Therefore, the 40 μg eGMCSF-HAₚ dose was selected for the consecutive in vivo studies.

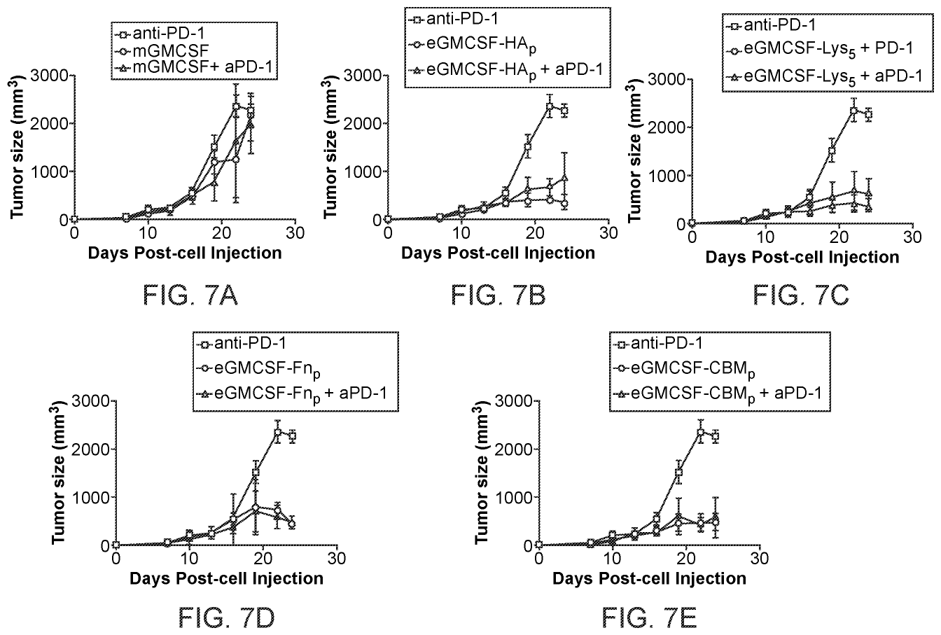

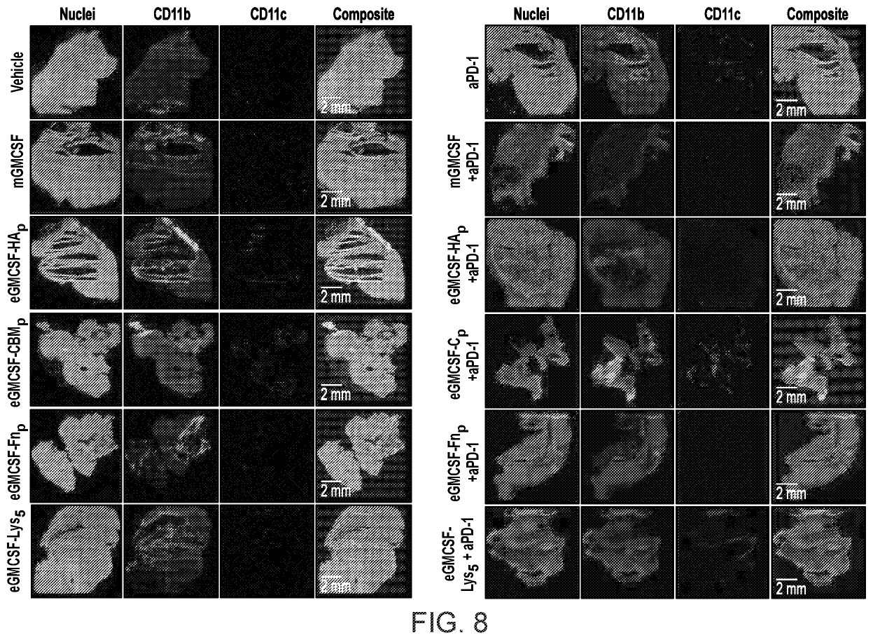

Upon establishing a safe working dose, the efficacy of eGMCSFs was investigated as monotherapy and in combination with the CPI anti-PD1. Mice treated with unmodified mGMCSF, anti-PD-1, and 4% mannitol served as controls. Similar to the previous study, colon-cancer mice bearing mice received IT injections of eGMCSF twice weekly for a total of 5 injections. For the combination therapy study, anti-PD-1 was administered through intraperitoneal (i.p) injections to all mice. The eGMCSF variants displayed a 3-6 times suppression in tumor growth when compared to the mice treated with vehicle controls and mGMCSF both with and without anti-PD-1 (FIGS. 6C-6D). No statistical difference was observed between the eGMCSF tested as a monotherapy and as a combination therapy (FIGS. 7A-7E). By contrast, the animals that received the unmodified mGMCSF cytokine displayed faster tumor growth rates that were similar to the tumor progression seen in untreated mice (mannitol vehicle control). We concluded that the tumor-retentive engineered cytokines exhibited potent anti-tumor efficacy upon IT delivery in murine colon cancer models.

FIG. 8 demonstrates that animals treated with eGMCSF variant monotherapy or combination therapy with anti-PD1 exhibited in vivo immune cell infiltration in colon cancer tumors.

### Example 7: Preclinical Safety of eGMCSF Therapy

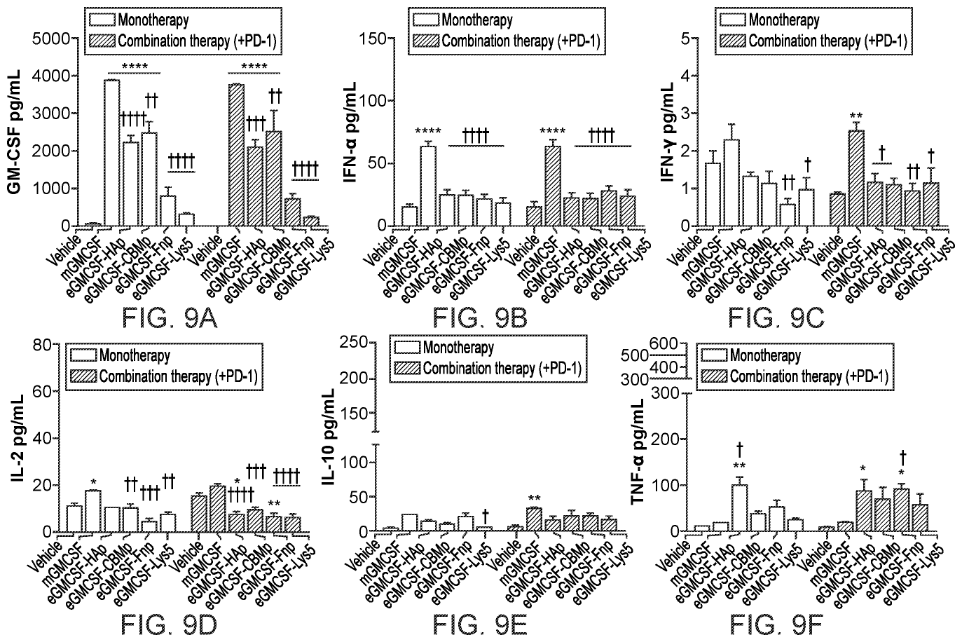

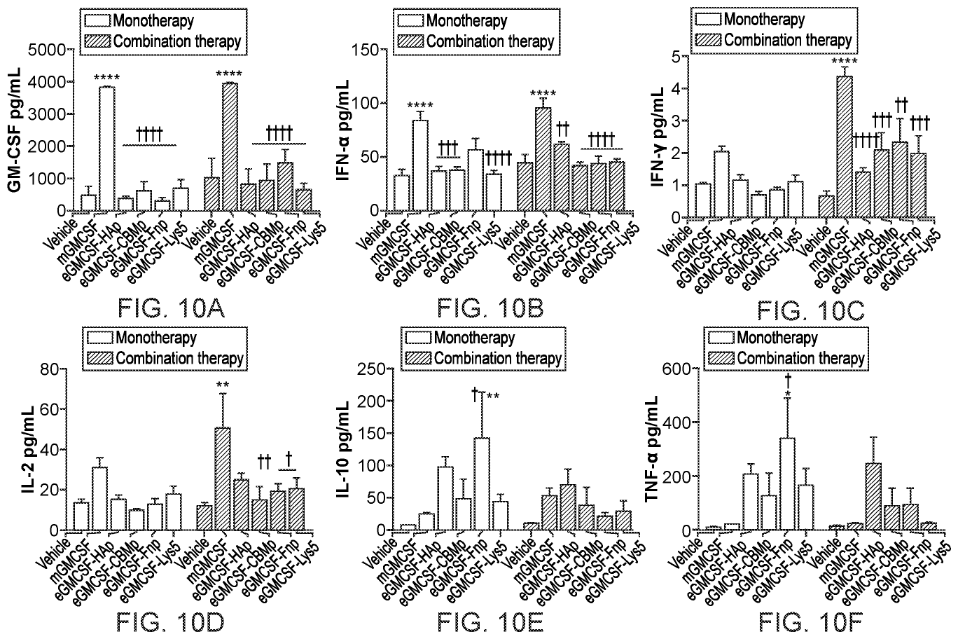

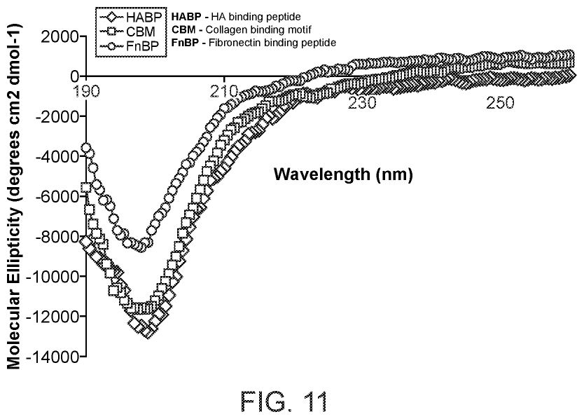

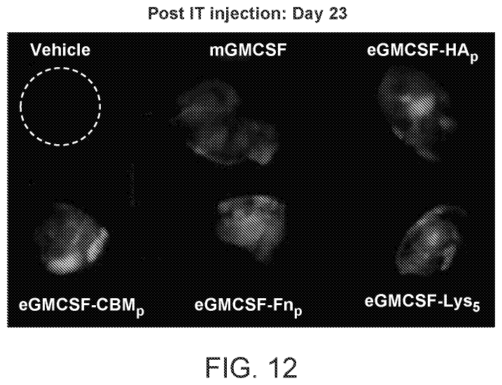

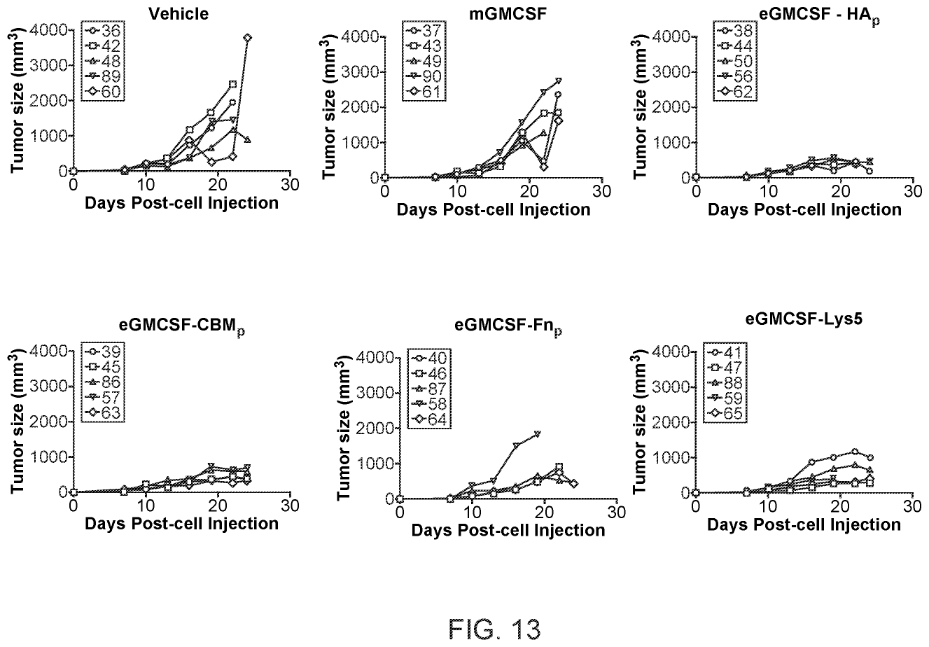

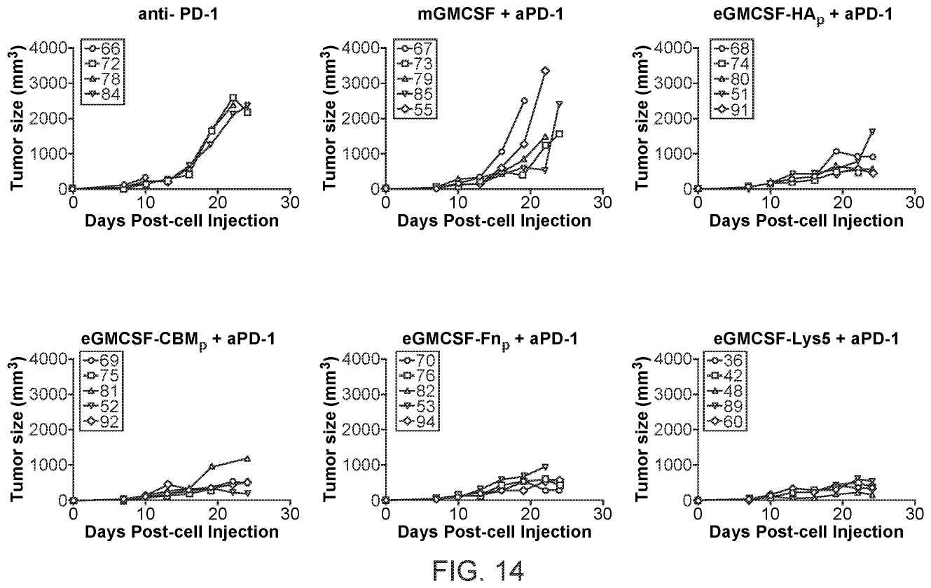

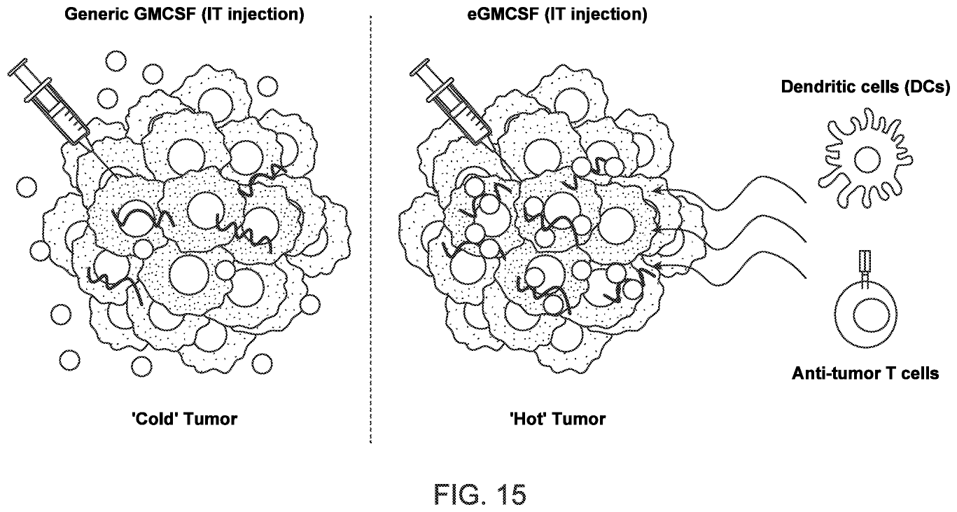

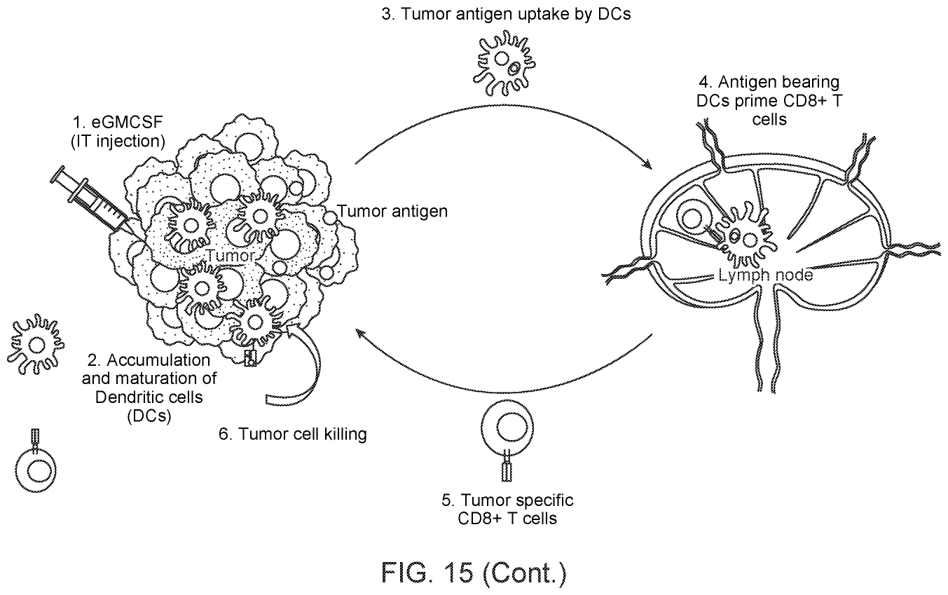

To evaluate the potential systemic toxicity of eGMCSF, pro-inflammatory cytokines (IL-10, IL-2, TNF-α, IFN-α, IFN-γ) were quantified.⁵³ Cytokine levels were determined from serum taken two hours after the first and last drug injections and mice treated with unmodified mGMCSF or mannitol vehicle served as controls. All mice treated with the different eGMCSF variants exhibited significantly lower levels of systemic cytokines IL-10, IL-2, IFN-α, IFN-γ in comparison to unmodified mGMCSF treatment groups. These cytokine levels were consistent between the monotherapy and combination therapy groups (FIGS. 9A-9E, FIGS. 10A-10E).

An opposite trend was observed with TNF-α, which exhibited increased serum levels after eGMCSF treatment (FIGS. 9F, 10F). TNF-α is a multifunctional cytokine produced by macrophages and has a paradoxical role in cancer.⁵⁸ These high levels in eGMCSF-treated mice could indicate anti-tumor responses, but a comprehensive study of the intratumoral environment would be necessary to draw firm conclusions.^{58, 59} Interestingly, the reduced levels of systemic GMCSF observed in the mice treated with eGMCSF reiterates the tumor-retentive nature of these recombinant proteins (FIGS. 9A & 10A). Overall, the reduced systemic pro-inflammatory cytokines levels are likely due to increased IT retention resulting in lower systemic toxicity.

GMCSF is typically selected as an immunostimulant due to its ability to activate DCs that are responsible for initiating cytotoxic T cell responses. Unfortunately, most cytokines have molecular properties that limit tumor retention and induce systemic immune-related adverse events (IRAEs). A short tumor residence time also limits immune activation in tumor tissue and fails to reverse the immunosuppressive environment (‘cold’ tumor).

The eGMCSF variants disclosed herein provide a design platform that can be adapted to impart tumor binding functionalities to other therapeutics. Built on the understanding of the tumor microenvironment, this study provides a design platform to develop effective protein therapeutics for the enhanced IT treatment of ‘cold’ tumors like breast, ovarian, prostate, pancreatic cancer, and glioblastomas.

We engineered a tumor-retentive version of GMCSF (eGMCSF) that persisted in tumor tissue to negate markers of systemic toxicity and elicit localized immune activation to effectively turn an unresponsive ‘cold’ tumor ‘hot’. This was attained by recombinantly fusing different tumor-ECM (extracellular matrix) binding peptides to GMCSF. Four eGMCSF variants were developed that bind to tumor ECM molecules like HA, collagen I, and fibronectin. Drug retention studies demonstrated the superior IT persistence of the eGMCSF formulations in murine models of head and neck cancer (HNSCC) and colon cancer compared to native GMCSF. The engineered drug variants had potent immunomodulatory activities as demonstrated by their proliferative effect on dendritic cells (DCs). Most notably, eGMCSF variants showed increased therapeutic efficacy in regressing tumor growth in comparison to unmodified mGMCSF and anti-PD-1 treatments in murine colon cancer models.

Additionally, systemic cytokines associated with immune toxicity were dramatically reduced in mice treated with the eGMCSF proteins when compared to native mGMCSF. Due to their ability to remain at the injection and induce local immune-cell infiltration, eGMSCF may work synergistically with check point inhibitors (CPIs), which are ineffective in the 70-80% of patients with a ‘cold’ tumor.

### REFERENCES

1. A. Huang, M. M. Pressnall, R. Lu, S. G. Huayamares, J. D. Griffin, C. Groer, B. J. DeKosky, M. L. Forrest and C. J. Berkland, J Control Release, 2020, 326, 203-221.
2. Y. Zhang and Z. Zhang, Cellular & Molecular Immunology, 2020, 17, 807-821.
3. P. Bonaventura, T. Shekarian, V. Alcazer, J. Valladeau-Guilemond, S. Valsesia-Wittmann, S. Amigorena, C. Caux and S. Depil, Front Immunol, 2019, 10.
4. Y.-T. Liu and Z.-J. Sun, Theranostics, 2021, 11, 5365-5386.
5. T. Pham, S. Roth, J. Desai, R. Ramsay and A. Heriot, Annals of Surgical Oncology, 2018, 25, 974-975.
6. N. Momin, J. R. Palmeri, E. A. Lutz, N. Jailkhani, H. Mak, A. Tabet, M. M. Chinn, B. H. Kang, V. Spanoudaki, R. O. Hynes and K. D. Wittrup, Nat Commun, 2022, 13, 109.
7. I. Melero, E. Castanon, M. Alvarez, S. Champiat and A. Marabelle, Nature Reviews Clinical Oncology, 2021, 18, 558-576.
8. Q. Duan, H. Zhang, J. Zheng and L. Zhang, Trends in Cancer, 2020, 6, 605-618.
9. M. Arellano and S. Lonial, Biologics, 2008, 2, 13-27.
10. H. M. Lazarus, C. E. Ragsdale, R. P. Gale and G. H. Lyman, Front Immunol, 2021, 12, 706186.
11. M. J. Mastrangelo, H. C. Maguire, L. C. Eisenlohr, C. E. Laughlin, C. E. Monken, P. A. McCue, A. J. Kovatich and E. C. Lattime, Cancer Gene Therapy, 1999, 6, 409-422.
12. C. Hoeller, B. Jansen, E. Heere-Ress, T. Pustelnik, U. Mossbacher, H. Schlagbauer-12. Wadl, K. Wolff and H. Pehamberger, Journal of Investigative Dermatology, 2001, 117, 371-374.
13. H. L. Kaufman, C. E. Ruby, T. Hughes and C. L. Slingluff, Journal for Immuno Therapy of Cancer, 2014, 2, 11.
14. P. F. Ferrucci, L. Pala, F. Conforti and E. Cocorocchio, Cancers (Basel), 2021, 13, 1383.
15. S. S. Raman, J. R. Hecht and E. Chan, Immunotherapy, 2019, 11, 705-723.
16. M. E. Mummert, M. Mohamadzadeh, D. I. Mummert, N. Mizumoto and A. Takashima, J Exp Med, 2000, 192, 769-779.
17. X. Gao and M. J. Groves, Eur J Pharm Biopharm, 1998, 45, 275-284.
18. J. Y. Lee, J. E. Choo, Y. S. Choi, J. B. Park, D. S. Min, S. J. Lee, H. K. Rhyu, I. H. Jo, C. P. Chung and Y. J. Park, Biomaterials, 2007, 28, 4257-4267.
19. L. Richert, F. Boulmedais, P. Lavalle, J. Mutterer, E. Ferreux, G. Decher, P. Schaaf, J. C. Voegel and C. Picart, Biomacromolecules, 2004, 5, 284-294.
20. B. Peltanova, M. Raudenska and M. Masarik, Molecular Cancer, 2019, 18, 63.
21. E. Henke, R. Nandigama and S. Ergün, Frontiers in Molecular Biosciences, 2020, 6.
22. M.-S. Kim, S.-E. Ha, M. Wu, H. Zogg, C. F. Ronkon, M.-Y. Lee and S. Ro, Int J Mol Sci, 2021, 22, 9185.
23. A. Naba, K. R. Clauser, C. A. Whittaker, S. A. Carr, K. K. Tanabe and R. O. Hynes, BMC Cancer, 2014, 14, 518.
24. T. Oskarsson, The Breast, 2013, 22, S66-S72.
25. D. A. Stewart, C. R. Cooper and R. A. Sikes, Reprod Biol Endocrinol, 2004, 2, 2-2.
26. V. M. Perez, J. F. Kearney and J. J. Yeh, Frontiers in Oncology, 2021, 11.
27. R. Malekian, S. Sima, A. Jahanian-Najafabadi, F. Moazen and V. Akbari, Protein Expr Purif, 2019, 160, 66-72.
28. J. F. DeLamarter, J. J. Mermod, C. M. Liang, J. F. Eliason and D. R. Thatcher, EMBO J, 1985, 4, 2575-2581.
29. C. K. Kim, C. H. Lee, S.-B. Lee and J.-W. Oh, PLOS One, 2013, 8, e80109.
30. C. A. Thomson, M. Olson, L. M. Jackson and J. W. Schrader, PLOS One, 2012, 7, e49891.
31. K. L. Maxwell, D. Bona, C. Liu, C. H. Arrowsmith and A. M. Edwards, Protein Sci, 2003, 12, 2073-2080.
32. P. Malyala and M. Singh, J Pharm Sci, 2008, 97, 2041-2044.
33 O. A. Ali, N. Huebsch, L. Cao, G. Dranoff and D. J. Mooney, Nature Materials, 2009, 8, 151-158.
34. K. D. Au-Apley, J. D. Au-Griffin, S. N. Au-Johnson, C. J. Au-Berkland and B. J. Au-DeKosky, JoVE, 2020, DOI: doi: 10.3791/61827, e61827.
35. F. Horkay, P. J. Basser, D. J. Londono, A.-M. Hecht and E. Geissler, J Chem Phys, 2009, 131, 184902-184902.
36. T. G. Park, J. H. Jeong and S. W. Kim, Adv Drug Deliv Rev, 2006, 58, 467-486.
37. S. G. Huayamares, J. Y. Song, A. Huang, S. R. Crowl, C. E. Groer, M. L. Forrest and C. J. Berkland, Macromolecular Bioscience, 2020, 20, 2000251.
38. G. Parmiani, C. Castelli, L. Pilla, M. Santinami, M. P. Colombo and L. Rivoltini, Ann Oncol, 2007, 18, 226-232.
39. D. Wang, W. Jiang, F. Zhu, X. Mao and S. Agrawal, Int J Oncol, 2018, 53, 1193-1203.
40. B. Li, M. VanRoey, C. Wang, T. H. Chen, A. Korman and K. Jooss, Clin Cancer Res, 2009, 15, 1623-1634.
41. A. Singh, V. Upadhyay, A. K. Upadhyay, S. M. Singh and A. K. Panda, Microbial Cell Factories, 2015, 14, 41.
42. A. Bhatwa, W. Wang, Y. I. Hassan, N. Abraham, X.-Z. Li and T. Zhou, Frontiers in Bioengineering and Biotechnology, 2021, 9.
43. J. C. Gasson, Blood, 1991, 77, 1131-1145.
44. D. M. Rogers, S. B. Jasim, N. T. Dyer, F. Auvray, M. Refrégiers and J. D. Hirst, Chem, 2019, 5, 2751-2774.
45. J. C. Gasson, Blood, 1991, 77, 1131-1145.
46. H. Lee, H. Fonge, B. Hoang, R. M. Reilly and C. Allen, Mol Pharm, 2010, 7, 1195-1208.
47. K. T. Xenaki, S. Oliveira and P. M. P. van Bergen en Henegouwen, Front Immunol, 2017, 8.
48. A. Schudel, D. M. Francis and S. N. Thomas, Nat Rev Mater, 2019, 4, 415-428.
49. E. Blanco, H. Shen and M. Ferrari, Nat Biotechnol, 2015, 33, 941-951.
50. W. Ngwa, O. C. Irabor, J. D. Schoenfeld, J. Hesser, S. Demaria and S. C. Formenti, Nat Rev Cancer, 2018, 18, 313-322.
51. R. Leary, R. B. Gardner, C. Mockbee and D. F. Roychowdhury, Cureus, 2019, 11, e4276-e4276.
52. L. van de Laar, P. J. Coffer and A. M. Woltman, Blood, 2012, 119, 3383-3393.
53. M. M. Pressnall, A. Huang, C. E. Groer, S. G. Huayamares, M. Laird Forrest and C. J. Berkland, International Journal of Pharmaceutics, 2021, 605, 120812.
54. J.-Y. Lee, J.-E. Choo, Y.-S. Choi, J.-B. Park, D.-S. Min, S.-J. Lee, H. K. Rhyu, I.-H. Jo, C.-P. Chung and Y.-J. Park, Biomaterials, 2007, 28, 4257-4267.
55. M. E. Mummert, M. Mohamadzadeh, D. I. Mummert, N. Mizumoto and A. Takashima, The Journal of experimental medicine, 2000, 192, 769-779.
56. C. M. Mantia and E. I. Buchbinder, Hematol Oncol Clin North Am, 2019, 33, 275-290.
57. S.-J. Lee, L. Song, M.-C. Yang, C.-P. Mao, B. Yang, A. Yang, J. Jeang, S. Peng, T. C. Wu and C.-F. Hung, Vaccine, 2015, 33, 1549-1555.
58. X. Wang and Y. Lin, Acta Pharmacol Sin, 2008, 29, 1275-1288.
59. A. Montfort, C. Colacios, T. Levade, N. Andrieu-Abadie, N. Meyer and B. Ségui, Front Immunol, 2019, 10.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A fusion polypeptide comprising a mammalian granulocyte macrophage-colony-stimulating factor (GMCSF) polypeptide operably linked to a tumor binding peptide, wherein the tumor binding peptide is configured to bind to a tumor extracellular matrix (ECM) component, wherein the tumor binding peptide comprises the amino acid sequence selected from the group consisting of SEQ ID NOs: 5-8 and 17-23.

2. The fusion polypeptide of claim 1, wherein the mammalian GMCSF polypeptide is murine GMCSF or human GMCSF; or
wherein the mammalian GMCSF polypeptide comprises an amino acid sequence selected from the group consisting of sargramostim, molgramostim, and regramostim; or
wherein the mammalian GMCSF polypeptide comprises an endogenous or heterologous signal peptide sequence; or
wherein the mammalian GMCSF polypeptide comprises the amino acid sequence of any one of SEQ ID NOs: 9, 13-16 or 24.

3. (canceled)

4. (canceled)

5. (canceled)

6. The fusion polypeptide of claim 1, wherein the mammalian GMCSF polypeptide is fused to the tumor binding peptide via a peptide linker, optionally wherein the peptide linker is a gly-ser polypeptide linker, a glycine-proline polypeptide linker, or a proline-alanine polypeptide linker.

7. (canceled)

8. The fusion polypeptide of claim 6, wherein the peptide linker is selected from the group consisting of S(G₄S)ₙ (SEQ ID NO: 25), (G₄S), (SEQ ID NO: 26), (G₃S)ₙ (SEQ ID NO: 27), (G₄S3)ₙ (SEQ ID NO: 28), (SG₄)ₙ (SEQ ID NO: 29) or G₄ (SG₄)ₙ (SEQ ID NO: 30), wherein n is 1, 2, 3, 4, 5, 6, 7, 8, 9, 10, 11, 12, 13, 14, or 15.

9. The fusion polypeptide of claim 1, wherein the tumor ECM component is hyaluronic acid, fibronectin, or collagen; or wherein the tumor binding peptide is linked to the N-terminus or C-terminus of the mammalian GMCSF polypeptide.

10. (canceled)

11. The fusion polypeptide of claim 1, wherein the tumor binding peptide comprises a collagen-binding domain, a hyaluronic acid binding peptide (HABP), integrin-binding polypeptide, or a fibronectin binding peptide (FnBP), optionally wherein the collagen-binding domain comprises a proteoglycan, optionally wherein the proteoglycan is selected from the group consisting of: decorin, biglycan, testican, bikunin, fibromodulin, lumican, chondroadherin, keratin, ECM2, epiphycan, asporin, PRELP, keratocan, osteoadherin, opticin, osteoglycan, nyctalopin, Tsukushi, podocan, podocan-like protein 1 versican, perlecan, nidogen, neurocan, aggrecan, osteopontin, and brevican.

12. (canceled)

13. The fusion polypeptide of claim 11, wherein the collagen-binding domain comprises a class I small leucine-rich proteoglycan (SLRP), a class II SLRP, a class III SLRP, a class IV SLRP, or a class V SLRP.

14. (canceled)

15. The fusion polypeptide of claim 1, comprising an amino acid sequence selected from the group consisting of SEQ ID NOs: 1-4.

16. A pharmaceutical composition comprising the fusion polypeptide of claim 1, and a pharmaceutically acceptable carrier.

17. A method for treating cancer or inhibiting tumor growth in a patient in need thereof, comprising administering to the patient a therapeutically effective amount of the fusion polypeptide of claim 1.

18. A method for enhancing responsiveness of a cancer patient to immune checkpoint inhibitor therapy comprising administering to the patient a therapeutically effective amount of the fusion polypeptide of claim 1; and administering to the patient a therapeutically effective amount of an immune checkpoint inhibitor.

19. The method of claim 17, wherein the cancer is a solid tumor.

20. The method of claim 18, wherein the cancer is melanoma, mesothelioma, pancreatic cancer, glioblastoma, breast cancer, ovarian cancer, lung cancer, colorectal cancer, or prostate cancer.

21. The method of claim 18, wherein the fusion polypeptide or the pharmaceutical composition is administered intratumorally, orally, topically, intranasally, systemically, intravenously, subcutaneously, intraperitoneally, intradermally, intraocularly, iontophoretically, transmucosally, or intramuscularly.

22. The method of claim 17, further comprising separately, sequentially or simultaneously administering one or more additional therapeutic agents to the subject.

23. The method of claim 22, wherein the one or more additional therapeutic agents comprise an immune checkpoint inhibitor, a chemotherapeutic agent and/or a radiotherapeutic agent.

24. The method of claim 23, wherein the immune checkpoint inhibitor is an anti-PD1 antibody or an anti-PD-L1 antibody.

25. The method of claim 18, wherein the immune checkpoint inhibitor comprises one or more of ipilimumab, pembrolizumab, nivolumab, atezolizumab, avelumab, durvalumab, pidilizumab, AMP-224, MPDL3280A, MDX-1105, MEDI-4736, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, or any combination thereof.

26. The method of claim 17, wherein administration of the fusion polypeptide or the pharmaceutical composition reduces the incidence of and/or mitigates systemic immune-related adverse events (IRAEs) in the patient.
